(19) United States
(12) Patent Application Publication
(10) Pub. No.: US 2003/0050306 A1
(43) Pub. Date: Mar. 13, 2003

(54) Novel heteroaryl derivatives, their preparation and use

(75) Inventors: Thomas Ruhland, Valby (DK); Christian Krog-Jensen, Copenhagen (DK); Ivan Mikkelsen, Koge (DK); Mario Rottlander, Valby (DK); Gitte Mikkelsen, Ballerup (DK); Ejner Knud Moltzen, Gentofte (DK); Kim Andersen, Virum (DK)

Correspondence Address: DARBY & DARBY P.C., 805 Third Avenue, New York, NY, 10022, US

(73) Assignee: H. Lundbeck A/S, Valby-Copenhagen (DK)

(21) Appl. No.: 10/183,957

(22) Filed: Jun. 25, 2002

Related U.S. Application Data
(63) Continuation of application No. PCT/DK00/00741, filed on Dec. 29, 2000.

(30) Foreign Application Priority Data
Dec. 30, 1999 (DK) PA 1999 01884

(51) Int. Cl. A61K 31/551; A61K 31/496; A61K 31/451; A61K 31/453; C 07D 4 9/02; C 07D 4 5/02

(52) U.S. Cl. 514/218; 514/252.13; 514/254.11; 540/575; 546/280.1; 546/282.7; 544/378; 514/321; 514/337; 546/196

(57) ABSTRACT

A heteroaryl derivative having the formula (I)

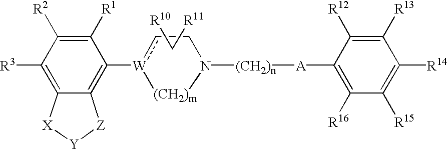

any of its enantiomers or any mixture thereof, wherein X is —O—, —S—, or CR⁴R⁵—; and Y is —CR⁶R⁷; —CR⁶R⁷—CR⁸R⁹—, or —CR⁶—CR⁷; or X and Y together form a group —CR⁴═R⁵—, or —CR⁴═CR⁵—CR⁶R⁷—; Z is —O—, or —S—; W is N, C, or CH; n is 2, 3, 4, 5, 6, 7, 8, 9 or 10; m is 2 or 3; A is O or S wherein the doted lines mean an optional bond. The compounds of the invention are considered useful for the treatment of affective disorders such as general anxiety disorder, panic disorder, obsessive compulsive disorder, depression, social phobia and eating disorders, and neurological disorders such as psychosis.

This application is a Continuation of International Application No. PCT/DK00/00741, filed Dec. 29, 2000. The disclosure of the prior application is hereby incorporated by reference.

The present invention relates to novel heteroaryl derivatives potently binding to the 5-HT_{1A} receptor, pharmaceutical compositions containing these compounds and the use thereof for the treatment of certain psychiatric and neurological disorders. The compounds of the invention are also potent dopamine D₄ receptor ligands and are considered to be particularly useful for the treatment of depression and psychosis.

Furthermore, many compounds of the invention have potent serotonin reuptake inhibition activity and/or effect at dopamine D₃ receptors.

### BACKGROUND ART

Clinical and pharmacological studies have shown that 5-HT_{1A} agonists and partial agonists are useful in the treatment of a range of affective disorders such as generalised anxiety disorder, panic disorder, obsessive compulsive disorder, depression and aggression.

It has also been reported that 5-HT_{1A} ligands may be useful in the treatment of ischaemia.

An overview of 5-HT_{1A} antagonists and proposed potential therapeutic targets for these antagonists based upon preclinical and clinical data are presented by Schechter et al., Serotonin , 1997, Vol.2, Issue 7. It is stated that 5-HT_{1A} antagonists may be useful in the treatment of schizophrenia, senile dementia, dementia associated with Alzheimer's disease, and in combination with SSRI antidepressants also to be useful in the treatment of depression.

5-HT reuptake inhibitors are well known antidepressant drugs and useful for the treatment of panic disorders and social phobia.

The effect of combined administration of a compound that inhibits serotonin reuptake and a 5-HT_{1A} receptor antagonist has been evaluated in several studies (Innis, R. B. et al., Eur. J. Pharmacol., 1987, 143, p 195-204 and Gartside, S. E., Br. J. Pharmacol. 1995, 115, p 1064-1070, Blier, P. et al, Trends Pharmacol. Sci. 1994, 15, 220). In these studies it was found that combined 5-HT_{1A} receptor antagonists and serotonin reuptake inhibitors would produce a more rapid onset of therapeutic action.

Dopamine D₄ receptors belong to the family of dopamine D₂ like receptors which is considered to be responsible for the antipsychotic effects of neuroleptics. Dopamine D₄ receptors are primarily located in areas of the brain other than striatum, suggesting that dopamine D₄ receptor ligands have antipsychotic effect and are devoid of extrapyramidal activity.

Accordingly, dopamine D₄ receptor ligands are potential drugs for the treatment of psychosis and positive symptoms of schizophrenia and compounds with combined effects at dopamine D₄, and serotonergic receptors may have the further benefit of improved effect on negative symptoms of schizophrenia, such as anxiety and depression, alcohol abuse, impulse control disorders, aggression, side effects induced by conventional antipsychotic agents, ischaemic disease states, migraine, senile dementia and cardiovascular disorders and in the improvement of sleep.

Dopamine D₃ receptors also belong to the family of dopamine D₂ like receptors. D₃ antagonistic properties of an antipsychotic drug could reduce the negative symptoms and cognitive deficits and result in an improved side effect profile with respect to EPS and hormonal changes.

Accordingly, agents acting on the 5-HT_{1A} receptor, both agonists and antagonists, are believed to be of potential use in the therapy of psychiatric and neurological disorders and thus being highly desired. Furthermore, antagonists at the same time having potent serotonin reuptake inhibition activity and/or D₄ and/or D₃ activity may be particularly useful for the treatment of various psychiatric and neurological diseases.

WO 95/04049 discloses related compounds of the general formula

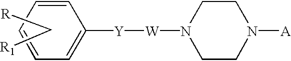

wherein A is a phenyl group or a benzofuran or benzodioxan group. These compounds are said to be α_{1A}-adrenergic receptor antagonists and to be useful for the prevention of contractions of the prostate, urethra and lower urinary tract

Bart J van Steen et al., Structure-Affinity Relationship Studies on 5-HT_{1A} receptor Ligands. 2. Heterobicyclic Phenylpiperazines with N4-Aralkyl Substituents, J. Med. Chem. ,1994, 37(17), 2761-73 describes certain related benzofuran and benzodioxan derivatives having affinity for the 5-HT_{1A} receptor and therefore being useful in the treatment of depression and anxiety.

### SUMMARY OF THE INVENTION

It has now been found that compounds of a certain class of heteroaryl derivatives bind to the 5-HT_{1A} receptor with high affinities. Additionally, the compounds also have effect at dopamine D₄ receptors. Furthermore, it has been found that many of the compounds have potent serotonin reuptake inhibition activity and/or effect at dopamine D₃ receptors.

Accordingly, the present invention relates to novel compounds of the general Formula I:

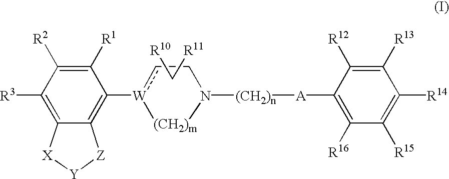

wherein

X is —O—, —S—, or —CR⁴R⁵—; and

Y is —CR⁶R⁷—, —CR⁶R⁷—CR⁸R⁹—, or —CR⁶═CR⁷—; or

X and Y together form a group —CR⁴═CR⁵—, or —CR⁴═CR⁵—CR⁶R⁷—;

Z is —O—, or —S—;

W is N, C, or CH;

n is 2, 3, 4, 5, 6, 7, 8, 9 or 10;

m is 2 or 3:

A is O or S

wherein the dotted lines mean an optional bond;

R¹, R² and R³ are each independently selected from hydrogen, halogen, nitro, cyano, trifluoromethyl, trifluoromethoxy, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylthio, hydroxy, formyl, acyl, amino, C₁₋₆-alkylamino, di(C₁₋₆-alkyl)amino, acylamino, C₁₋₆-alkoxycarbonylamino, aminocarbonylamino, C₁₋₆-alkylaminocarbonylamino and di(C₁₋₆-alkyl)aminocarbonylamino;

R⁴, R⁵, R⁶, R⁷, R⁸ and R⁹ are each independently selected from hydrogen, halogen, trifluoromethyl, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylthio, amino, C₁₋₆-alkylamino, di(C₁₋₆-alkyl)amino, phenylamino or phenyl-C₁₋₆-alkylamino wherein the phenyl group may be substituted, acylamino, hydroxy, —SH, cyano, nitro, —COOR¹⁸, —SO₂—R¹⁹ or

C₁₋₆-alkyl substituted with a substituent selected from halogen, C₁₋₆-alkoxy, C₁₋₆-alkylthio, amino, C₁₋₆-alkylamino, di(C₁₋₆-alkyl)amino, acylamino, hydroxy, —SH, cyano, nitro, —COOR¹⁸ or —SO₂—R¹⁹;

R¹⁸ is hydrogen, C₁₋₆-alkyl, C₂₋₆-alkenyl, C₂₋₆-alkynyl, phenyl or phenyl-C₁₋₆-alkyl wherein the phenyl groups may be substituted, amino, C₁₋₆-alkylamino or di(C₁₋₆-alkyl)amino, and

R¹⁹ is hydrogen, C₁₋₆-alkyl, amino, C₁₋₆-alkylamino, di(C₁₋₆-alkyl)amino, phenyl or phenyl-C₁₋₆-alkyl wherein the phenyl groups may be substituted;

R¹⁰ and R¹¹ are each independently selected from hydrogen and C₁₋₆-alkyl; and

R¹², R¹³, R¹⁴, R¹⁵ and R¹⁶ are each independently selected from hydrogen, halogen, nitro, cyano, trifluoromethyl, trifluoromethoxy, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylthio, C₁₋₆-alkylsulphonyl, hydroxy, formyl, acyl, amino, acylamino, C₁₋₆-alkoxycarbonylamino, aminocarbonylamino, C₁₋₆-alkylaminocarbonylamino, di(C₁₋₆-alkyl)aminocarbonylamino and NR²⁰R²¹ wherein R²⁰ and R²¹ independently represent hydrogen, C₁₋₆-alkyl, C₃₋₈-cycloalkyl, or phenyl; or R²⁰ and R²¹ together with the nitrogen to which they are attached form a 5- or 6-membered carbocyclic ring optionally containing one further heteroatom;

provided that when X-Y-Z together with the phenyl ring forms a benzofuran or a benzodioxan ring; and A is O, then at least one of R¹², R¹³, R¹⁴, R¹⁵ and R¹⁶ is not hydrogen;

any of its enantiomers or any mixture thereof, or an acid addition salt thereof.

In one embodiment of the invention X is —O—; and Y is —CR⁶R⁷—CR⁸R⁹—; and Z is —O—.

In another embodiment of the invention X is —CR⁴R⁵—; and Y is —CR⁶R⁷; and Z is —O—.

In a further of the invention X and Y together form a group —CR⁴═CR⁵—; and Z is —S—.

In a further embodiment of the invention A is O.

In a further embodiment of the invention A is S.

In a further embodiment of the invention W is N.

In a further embodiment of the invention R¹, R² and R³ are hydrogen;

In a further embodiment of the invention n is 2, 3 or 4;

In a further embodiment of the invention R¹², R¹³, R¹⁴, R¹⁵ and R¹⁶ are independently selected from the group consisting of hydrogen, halogen, C₁₋₆-alkyl, C₂₋₆-alkenyl, C₁₋₆-alkoxy, cyano, C₁₋₆-alkylsulphonyl, acyl, nitro, trifluoromethyl, and trifluoromethxoy.

In a preferred embodiment of the invention at least one of R¹², R¹³, R¹⁴, R¹⁵ and R¹⁶ is halogen.

In a further preferred embodiment of the invention at least one of R¹², R¹³, R¹⁴, R¹⁵ and R¹⁶ is halogen. and the other substituents are selected from the group consisting of hydrogen, halogen, C₁₋₆-alkoxy, C₁₋₆-alkyl, C₂₋₆-alkenyl, C₁₋₆-alkylsulfonyl, acyl, nitro, cyano and trifluoromethyl;

Specific compounds of the invention are compounds selected from

1-[3-(2-Chloro-phenoxy)-propyl]-4-(2,3-dihydro-benzo[1,4]dioxin-5-yl)-piperazine;

1-[3-(2,6-Dichloro-phenoxy)-propyl]-4-(2,3-dihydro-benzo[1,4]dioxin-5-yl)-piperazine;

1-(2,3-Dihydro-benzo[1,4]dioxin-5-yl)-4-[3-(2,4,6-trifluoro-phenoxy)-propyl]-piperazine;

1-(2,3-Dihydro-benzo[1,4]dioxin-5-yl)-4-[3-(4-fluoro-2-methoxy-phenoxy)-propyl]-piperazine;

1-(2,3-Dihydro-benzo[1,4]dioxin-5-yl)-4-[3-(4-fluoro-2-methyl-phenoxy)-propyl]-piperazine;

1-[3-(4-Chloro-phenoxy)-propyl]-4-(2,3-dihydro-benzo[1,4]dioxin-5-yl)-piperazine;

1-(2,3-Dihydro-benzo[1,4]dioxin-5-yl)-4-[3-(4-trifluoromethyl-phenoxy)-propyl]-piperazine;

1-(2,3-D ihydro-benzo[1,4]dioxin-5-yl)-4-[3-(2-fluoro-phenoxy)-propyl]-piperazine;

2-{3-[4-(2,3-Dihydro-benzo[1,4]dioxin-5-yl)-piperazin-1-yl]-propoxy}-benzonitrile;

1-Benzo[b]thiophen-7-yl-4-[3-(2-chloro-4-fluoro-phenylsulfanyl)-propyl]-piperazine;

1-Benzo[b]thiophen-7-yl-4-[4-(2-chloro-4-fluoro-phenoxy)-butyl]-piperazine;

1-[2-(3,4-Dichloro-phenylsulfanyl)-ethyl]-4-(2,3-dihydro-benzo[1,4]dioxin-5-yl)-piperazine;

1-(2,3-Dihydro-benzo[1,4]dioxin-5-yl)-4-[2-(4-fluoro-phenylsulfanyl)-ethyl]-piperazine;

1-[2-(Bromo-trifluoromethyl-phenylsulfanyl)-ethyl]-4-(2,3-dihydro-benzo[1,4]dioxin-5-yl)-piperazine;

1-[2-(2,6-Dichloro-phenylsulfanyl)-ethyl]-4-(2,3-dihydro-benzo[1,4]dioxin-5-yl)-piperazine;

1-(2,3-Dihydro-benzo[1,4]dioxin-5-yl)-4-(3-phenylsulfanyl-propyl)-piperazine;

1-[3-(2-Bromo-4-fluoro-phenoxy)-propyl]-4-(2,3-dihydro-benzo[1,4]dioxin-5-yl)-piperazine;

1-[4-(2,6-Dichloro-phenylsulfanyl)-butyl]-4-(2,3-dihydro-benzol[1,4]dioxin-5-yl)-piperazine;

1-[3-(2-Chloro-4-fluoro-phenylsulfanyl)-propyl]-4-(2,3-dihydro-benzo[1,4]dioxin-5-yl)-piperazine;

1-Benzo[b]thiophen-7-yl-4-[3-(2-chloro-4-fluoro-phenylsulfanyl)-propyl]-piperazine;

1-Benzo[b]thiophen-7-yl-4-[3-(2,6-dichloro-phenylsulfanyl)-propyl]-piperazine;

1-Benzo[b]thiophen-7-yl-4-[4-(2,6-dichloro-phenylsulfanyl)-butyl]-piperazine;

1-[4-(3-Chloro-2-methoxy-phenylsulfanyl)-butyl]-4-(2,3-dihydro-benzo[1,4]dioxin-5-yl)-piperazine;

1-Benzo[b]thiophen-7-yl-4-[4-(3-chloro-2-methoxy-phenylsulfanyl)-butyl]-piperazine;

1-Benzo[b]thiophen-7-yl-4-[3-(2-chloro-4-fluoro-phenylsulfanyl)-propyl]-piperazine;

1-[3-(2,6-Dibromo-4-fluoro-phenoxy)-propyl]-4-(2,3-dihydro-benzo[1,4]dioxin-5-yl)-piperazine;

1-Benzo[b]thiophen-7-yl-4-[3-(2,6-dibromo-4-fluoro-phenoxy)-propyl]-piperazine;

4-{3-[4-(2,3-Dihydro-benzo[1,4]dioxin-5-yl)-piperazin-1-yl]-propoxy}-3,5-diiodo-benzonitrile;

3,5-Di-tert-butyl-4-{3-[4-(2,3-dihydro-benzo[1,4]dioxin-5-yl)-piperazin-1-yl]-propoxy}-benzonitrile;

1-[3-(2,6-Dichloro-4-methanesulfonyl-phenoxy)-propyl]-4-(2,3-dihydro-benzo[1,4]dioxin-5-yl)-piperazine;

1-Benzo[b]thiophen-7-yl-4-[3-(2,6-dichloro-4-methanesulfonyl-phenoxy)-propyl]-piperazine;

1-[3-(Bromo-trifluoromethyl-phenylsulfanyl)-propyl]-4-(2,3-dihydro-benzo[1,4]dioxin-5-yl)-piperazine;

1-Benzo[b]thiophen-7-yl-4-[3-(bromo-trifluoromethyl-phenylsulfanyl)-propyl]-piperazine;

1-Benzo[b]thiophen-7-yl-4-[4-(2-chloro-6-methyl-phenylsulfanyl)-butyl]-piperazine;

1-Benzo[b]thiophen-7-yl-4-[4-(2-chloro-4-fluoro-phenylsulfanyl)-butyl]-piperazine;

1-[3-(2,6-Dichloro-4-fluoro-phenoxy)-propyl]-4-(2,3-dihydro-benzo[1,4]dioxin-5-yl)-piperazine;

1-Benzo[b]thiophen-7-yl-4-[3-(2,6-dichloro-4-fluoro-phenoxy)-propyl]-piperazine;

1-[4-(2-Chloro-6-methyl-phenylsulfanyl)-butyl]-4-(2,3-dihydro-benzo[1,4]dioxin-5-yl)-piperazine;

1-[3-(2,6-Dichloro-phenylsulfanyl)-propyl]-4-(2,3-dihydro-benzo[1,4]dioxin-5-yl)-piperazine;

1-(5-Chloro-2,2-dimethyl-2,3-dihydro-benzofuran-7-yl)-4-[4-(2-chloro-6-methyl-phenylsulfanyl)-butyl]-piperazine;

1-(5-Chloro-3,3-dimethyl-2,3-dihydro-benzofuran-7-yl)-4-[4-(2-chloro-6-methyl-phenylsulfanyl)-butyl]-piperazine;

1-(5-Chloro-2,2-dimethyl-2,3-dihydro-benzofuran-7-yl)-4-[3-(2,6-dichloro-phenylsulfanyl)-propyl]-piperazine;

1-(5-Chloro-3,3-dimethyl-2,3-dihydro-benzofuran-7-yl)-4-[3-(2,6-dichloro-phenysulfanyl)-propyl]-piperazine;

1-(5-Chloro-3,3-dimethyl-2,3-dihydro-benzofuran-7-yl)-4-[3-(2-chloro-4-fluoro-phenylsulfanyl)-propyl]-piperazine;

1-Benzo[b]thiophen-7-yl-4-[4-(2-chloro-4-fluoro-phenoxy)-butyl]-piperazine;

1-(5-Chloro-2,2-dimethyl-2,3-dihydro-benzofuran-7-yl)-4-[4-(2-chloro-4-fluoro-phenoxy)-butyl]-piperazine;

1-[4-(2-Bromo-4-fluoro-phenoxy)-butyl]-4-(2,3-dihydro-benzo[1,4]dioxin-5-yl)-piperazine;

1-Benzo[b]thiophen-7-yl-4-[4-(2-bromo-4-fluoro-phenoxy)-butyl]-piperazine;

1-[4-(2-Bromo-4-fluoro-phenoxy)-butyl]-4-(5-chloro-2,2-dimethyl-2,3-dihydro-benzofuran-7-yl)-piperazine;

1-(5-Chloro-2,2-dimethyl-2,3-dihydro-benzofuran-7-yl)-4-[3-(2,6-dichloro-4-methanesulfonyl-phenoxy)-propyl]-piperazine;

1-(5-Chloro-3,3-dimethyl-2,3-dihydro-benzofuran-7-yl)-4-[3-(2,6-dichloro-4-methanesulfonyl-phenoxy)-propyl]-piperazine;

1-(5-Chloro-3,3-dimethyl-2,3-dihydro-benzofuran-7-yl)-4-[4-(3-chloro-2-methoxy-phenylsulfanyl)-butyl]-piperazine;

1-(5-Chloro-2,2-dimethyl-2,3-dihydro-benzofuran-7-yl)-4-[3-(2,6-dichloro-4-fluoro-phenoxy)-propyl]-piperazine;

1-(5-Chloro-3,3-dimethyl-2,3-dihydro-benzofuran-7-yl)-4-[3-(2,6-dichloro-4-fluoro-phenoxy)-propyl]-piperazine;

1-(4-{4-[4-(2,3-Dihydro-benzo[1,4]dioxin-5-yl)-piperazin-1-yl]-butoxy}-3,5-difluoro-phenyl)-propan-1-one;

1-[2-(2-Bromo-4,6-difluoro-phenoxy)-ethyl]-4-(2,3-dihydro-benzo[1,4]dioxin-5-yl)-piperazine;

1-[3-(2-Bromo-4,6-difluoro-phenoxy)-propyl]-4-(2,3-dihydro-benzo[1,4]dioxin-5-yl)-piperazine;

1-[4-(2,6-Dichloro-4-fluoro-phenoxy)-butyl]-4-(2,3-dihydro-benzo[1,4]dioxin-5-yl)-piperazine;

1-(2,3-Dihydro-benzo[1,4]dioxin-5-yl)-4-[3-(2,4,6-tribromo-phenoxy)-propyl]-piperazine;

1-(4-{3-[4-(2,3-Dihydro-benzo[1,4]dioxin-5-yl)-piperazin-1-yl]-propoxy}-3,5-difluoro-phenyl)-propan-1-one;

1-{4-[4-(4-Benzo[b]thiophen-7-yl-piperazin-1-yl)-butoxy]-3,5-difluoro-phenyl}-propan-1-one;

1-Benzo[b]thiophen-7-yl-4-[3-(2-bromo-4,6-difluoro-phenoxy)-propyl]-piperazine;

1-Benzo[b]thiophen-7-yl-4-[4-(2,6-dichloro-4-fluoro-phenoxy)-butyl]-piperazine;

1-Benzo[b]thiophen-7-yl-4-[3-(2,4,6-tribromo-phenoxy)-propyl]-piperazine;

1-{4-[3-(4-Benzo[b]thiophen-7-yl-piperazin-1-yl)-propoxy]-3,5-difluoro-phenyl}-propan-1-one;

3,5-Dibromo-4-{3-[4-(2,3-dihydro-benzo[1,4]dioxin-5-yl)-piperazin-1-yl]-propoxy}-benzonitrile;

1-[4-(2,6-Dibromo-4-fluoro-phenoxy)-butyl]-4-(2,3-dihydro-benzo[1,4]dioxin-5-yl)-piperazine;

1-[4-(4-Bromo-2,6-difluoro-phenoxy)-butyl]-4-(2,3-dihydro-benzo[1,4]dioxin-5-yl)-piperazine;

1-Benzo[b]thiophen-7-yl-4-[3-(2,6-dibromo-4-nitro-phenoxy)-propyl]-piperazine;

4-[3-(4-Benzo[b]thiophen-7-yl-piperazin-1-yl)-propoxy]-3,5-dibromo-benzonitrile;

1-Benzo[b]thiophen-7-yl-4-[4-(4-bromo-2,6-difluoro-phenoxy)-butyl]-piperazine;

1-[3-(2-Chloro-phenylsulfanyl)-propyl]-4-(2,3-dihydro-benzo[1,4]dioxin-5-yl)-piperazine;

1-Benzo[b]thiophen-7-yl-4-[3-(2-chloro-phenylsulfanyl)-propyl]-piperazine;

1-[3-(2,4-Difluoro-phenoxy)-propyl]-4-(2,3-dihydro-benzo[1,4]dioxin-5-yl)-piperazine;

25 1-[3-(4-Bromo-2,6-difluoro-phenoxy)-propyl]-4-(2,3-dihydro-benzo[1,4]dioxin-5-yl)-piperazine;

1-Benzo[b]thiophen-7-yl-4-[2-(2-bromo-4,6-difluoro-phenoxy)-ethyl]-piperazine;

1-Benzo[b]thiophen-7-yl-4-[3-(2,4-difluoro-phenoxy)-propyl]-piperazine;

1-Benzo[b]thiophen-7-yl-4-[3-(4-bromo-2,6-difluoro-phenoxy)-propyl]-piperazine;

8-{4-[3-(2-chloro-4-fluorophenoxy)-propyl]-piperazin-1-yl}-2,3-dihydro-benzo[1,4]dioxine-5-carbonitrile;

8-{4-[3-(2,6-Dichloro-phenoxy)-propyl]-piperazin-1-yl}-2,3-dihydro-benzo[1,4]dioxine-5-carbonitrile;

8-{4-[3-(4-Fluoro-2-methyl-phenoxy)-propyl]-piperazin-1-yl}-2,3-dihydro-benzo[1,4]dioxine-5-carbonitrile;

8-{4-[3-(2-Bromo-4-fluoro-phenoxy)-propyl]-piperazin-1-yl}-2,3-dihydro-benzo[1,4]dioxine-5-carbonitrile;

8-{4-[3-(2-Chloro-phenoxy)-propyl]-piperazin-1-yl}-2,3-dihydro-benzo[1,4]dioxine-5-carbonitrile;

1-(2,3-Dihydro-benzo[1,4]dioxin-5-yl)-4-(2-phenylsulfanyl-ethyl)-piperazine;

1-(2,3-Dihydro-benzo[1,4]dioxin-5-yl)-4-[2-(2,6-dimethyl-phenoxy)-ethyl]-piperazine;

1-(2,3-Dihydro-benzo[1,4]dioxin-5-yl)-4-[4-(2,6-dimethyl-phenylsulfanyl)-butyl]-piperazine;

1-(2,3-Dihydro-benzo[1,4]dioxin-5-yl)-4-[2-(2,4-dimethyl-phenylsulfanyl)-ethyl]-piperazine;

1-(2,3-Dihydro-benzo[1,4]dioxin-5-yl)-4-[2-(2-trifluoromethyl-phenoxy)-ethyl]-piperazine;

1-(2,3-Dihydro-benzo[1,4]dioxin-5-yl)-4-[2-(2-trifluoromethyl-phenylsulfanyl)-ethyl]-piperazine;

1-(2,3-Dihydro-benzo[1,4]dioxin-5-yl)-4-[2-(2-ethyl-phenoxy)-ethyl]-piperazine;

1-[2-(2,3-Dichloro-phenylsulfanyl)-ethyl]-4-(2,3-dihydro-benzo[1,4]dioxin-5-yl)-piperazine;

1-[2-(2-Allyl-6-chloro-phenoxy)-ethyl]-4-(2,3-dihydro-benzo[1,4]dioxin-5-yl)-piperazine;

1-(2,3-Dihydro-benzo[1,4]dioxin-5-yl)-4-[3-(2,4-dimethyl-phenylsulfanyl)-propyl]-piperazine;

1-(2,3-Dihydro-benzo[1,4]dioxin-5-yl)-4-[3-(2-trifluoromethyl-phenylsulfanyl)-propyl]-piperazine;

1-[3-(2,3-Dichloro-phenylsulfanyl)-propyl]-4-(2,3-dihydro-benzo[1,4]dioxin-5-yl)-piperazine;

1-[3-(3,4-Dichloro-phenylsulfanyl)-propyl]-4-(2,3-dihydro-benzo[1,4]dioxin-5-yl)-piperazine;

1-[4-(3,4-Dichloro-phenylsulfanyl)-butyl]-4-(2,3-dihydro-benzo[1,4]dioxin-5-yl)-piperazine;

1-[4-(2-Chloro-5-methyl-phenoxy)-butyl]-4-(2,3-dihydro-benzo[1,4]dioxin-5-yl)-piperazine;

1-[2-2,4-Dichloro-phenylsulfanyl)-ethyl]-4-(2,3-dihydro-benzo[1,4]dioxin-5-yl)-piperazine;

1-(2,3-Dihydro-benzo[1,4]dioxin-5-yl)-4-(3-m-tolylsulfanyl-propyl)-piperazine;

1-[4-(2,4-Dichloro-phenylsulfanyl)-butyl]-4-(2,3-dihydro-benzo[1,4]dioxin-5-yl)-piperazine;

1-(2,3-Dihydro-benzo[1,4]dioxin-5-yl)-4-[2-(2-ethyl-phenylsulfanyl)-ethyl]-piperazine;

1-[2-(2,5-Dichloro-phenylsulfanyl)-ethyl]-4-(2,3-dihydro-benzo[1,4]dioxin-5-yl)-piperazine;

1-[2-(3-Chloro-phenylsulfanyl)-ethyl]-4-(2,3-dihydro-benzo[1,4]dioxin-5-yl)-piperazine;

1-[2-(2-Chloro-phenylsulfanyl)-ethyl]-4-(2,3-dihydro-benzo[1,4]dioxin-5-yl)-piperazine;

1-(2,3-Dihydro-benzo[1,4]dioxin-5-yl)-4-[2-(2-fluoro-phenylsulfanyl)-ethyl]-piperazine;

1-(2,3-Dihydro-benzo[1,4]dioxin-5-yl)-4-[3-(2-ethyl-phenylsulfanyl)-propyl]-piperazine;

1-[3-(2,5-Dichloro-phenylsulfanyl)-propyl]-4-(2,3-dihydro-benzo[1,4]dioxin-5-yl)-piperazine;

1-[3-(3-Chloro-phenylsulfanyl)-propyl]-4-(2,3-dihydro-benzo[1,4]dioxin-5-yl)-piperazine;

1-(2,3-Dihydro-benzo[1,4]dioxin-5-yl)-4-[3-(2-fluoro-phenylsulfanyl)-propyl]-piperazine;

3-Chloro-4-{4-[4-(2,3-dihydro-benzo[1,4]dioxin-5-yl)-piperazin-1-yl]-butoxy}-benzonitrile;

1-(2,3-Dihydro-benzo[1,4]dioxin-5-yl)-4-(4-o-tolylsulfanyl-butyl)-piperazine;

1-[4-(2,5-Dichloro-phenylsulfanyl)-butyl]-4-(2,3-dihydro-benzo[1,4]dioxin-5-yl)-piperazine;

1-[4-(2-Chloro-phenylsulfanyl)-butyl]-4-(2,3-dihydro-benzo[1,4]dioxin-5-yl)-piperazine;

1-(2,3-Dihydro-benzo[1,4]dioxin-5-yl)-4-[4-(2-fluoro-phenylsulfanyl)-butyl]-piperazine;

1-(2,3-Dihydro-benzo[1,4]dioxin-5-yl)-4-[2-(3,4-dimethoxy-phenylsulfanyl)-ethyl]-piperazine;

3-{4-[4-(2,3-Dihydro-benzo[1,4]dioxin-5-yl)-piperazin-1-yl]-butoxy}-benzonitrile;

1-[4-(2-Chloro-4-fluoro-phenylsulfanyl)-butyl]-4-(2,3-dihydro-benzo[1,4]dioxin-5-yl)-piperazine;

1-(2,3-Dihydro-benzo[1,4]dioxin-5-yl)-4-[3-(4-trifluoromethoxy-phenylsulfanyl)-propyl]-piperazine;

1-(2,3-Dihydro-benzo[1,4]dioxin-5-yl)-4-[3-(2,5-dimethoxy-phenylsulfanyl)-propyl]-piperazine;

1-(2,3-Dihydro-benzo[1,4]dioxin-5-yl)-4-[3-(3-bromo-phenylsulfanyl)-propyl]-piperazine;

1-(2,3-Dihydro-benzo[1,4]dioxin-5-yl)-4-[4-(2-methoxy-phenylsulfanyl)-butyl]-piperazine;

1-(2,3-Dihydro-benzo[1,4]dioxin-5-yl)-4-[4-(2-isopropyl-phenylsulfanyl)-butyl]-piperazine;

1-(2,3-Dihydro-benzo[1,4]dioxin-5-yl)-4-(2-o-tolylsulfanyl-ethyl)-piperazine;

1-[4-(2-Allyl-phenoxy)-butyl]-4-(2,3-dihydro-benzo[1,4]dioxin-5-yl)-piperazine;

or an acid addition salt thereof.

The invention also relates to a pharmaceutical composition comprising a compound of formula (I) or a pharmaceutically acceptable salt thereof and at least one pharmaceutically acceptable carrier or diluent.

In a further embodiment, the invention relates to the use of a compound of formula (I) or a pharmaceutically acceptable acid addition salt thereof for the preparation of a medicament for the treatment of a disorder or disease responsive to the combined effect of 5-HT_{1A} receptors and dopamine D₄ receptors.

In a further embodiment, the invention relates to the use of a compound of formula (I) or a pharmaceutically acceptable acid addition salt thereof for the preparation of a medicament for the treatment of a disorder or disease responsive to the inhibition of serotonin uptake and antagonism of 5-HT_{1A} receptors.

In particular, the invention relates to the use of a compound according to the invention or a pharmaceutically acceptable acid addition salt thereof for the preparation of a medicament for the treatment of affective disorders such as general anxiety disorder, panic disorder, obsessive compulsive disorder, depression, social phobia and eating disorders, and neurological disorders such as psychosis.

In still another embodiment, the present invention relates to a method for the treatment of a disorder or disease of living animal body, including a human, which is responsive to the effect of 5-HT_{1A} and D₄ receptors comprising administering to such a living animal body, including a human, a therapeutically effective amount of a compound of formula (I) or a pharmaceutically acceptable acid addition salt thereof.

The compounds of the invention have high affinity for the 5-HT_{1A} and D₄ receptors. Accordingly, the compounds of the invention are considered useful for the treatment of affective disorders such as general anxiety disorder, panic disorder, obsessive compulsive disorder, depression, social phobia and eating disorders, and neurological disorders such as psychosis.

Due to their combined antagonism of 5-HT_{1A} receptors and serotonin reuptake inhibiting effect, many of the compounds of the invention are considered particularly useful as fast onset of action medicaments for the treatment of depression. The compounds may also be useful for the treatment of depression in patients who are resistant to treatment with currently available antidepressants.

### DETAILED DESCRIPTION OF THE INVENTION

Some of the compounds of general Formula I may exist as optical isomers thereof and such optical isomers are also embraced by the invention.

The term C₁₋₆ alkyl refers to a branched or unbranched alkyl group having from one to six carbon atoms inclusive, such as methyl, ethyl, 1-propyl, 2-propyl, 1-butyl, 2-butyl, 2-methyl-2-propyl and 2-methyl-1-propyl.

Similarly, C₂₋₆ alkenyl and C₂₋₆ alkynyl, respectively, designate such groups having from two to six carbon atoms, inclusive.

Halogen means fluoro, chloro, bromo, or iodo.

The term C₃₋₈ cycloalkyl designates a monocyclic or bicyclic carbocycle having three to eight C-atoms, such as cyclopropyl, cyclopentyl, cyclohexyl, cycloheptyl, and cyclooctyl.

The terms C₁₋₆ alkoxy, C₁₋₆ alkylthio and C₁₋₆ alkylsulphonyl designate such groups in which the alkyl group is C₁₋₆ alkyl as defined above.

Acyl means —CO-alkyl wherein the alkyl group is C₁₋₆ alkyl as defined above.

Amino means NH₂.

C₁₋₆ alkylamino means —NH-alkyl, and di(C₁₋₆-alkyl)amino means —N-(alkyl)₂ where the alkyl group is C₁₋₆ alkyl as defined above.

Acylamino means —NH-acyl wherein acyl is as defined above.

C₁₋₆ alkoxycarbonylamino means alkyl-O—CO—NH— wherein the alkyl group is C₁₋₆ alkyl as defined above.

C₁₋₆ alkylaminocarbonylamino means alkyl-NH—CO—NH— wherein the alkyl group is C₁₋₆ alkyl as defined above.

di(C₁₋₆-alkyl)aminocarbonylamino means (alkyl)₂—N—CO—NH— wherein the alkyl group is C₁₋₆alkyl as defined above.

As used herein, a phenyl group which may be substituted means a phenyl group which may be substituted one or more times with a substituent selected form halogen, trifluoromethyl, cyano, nitro, amino, C₁₋₆-alkylamino, di(C₁₋₆-alkyl)amino, C₁₋₆-alkyl, C₁₋₆-alkoxy and hydroxy.

Exemplary of organic acid addition salts according to the invention are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of inorganic acid addition salts according to the invention are those with hydrochloric, hydrobromic, sulfuric, sulfamic, phosphoric, and nitric acids. The acid addition salts of the invention are preferably pharmaceutically acceptable salts formed with non-toxic acids.

Furthermore, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

Some of the compounds of the present invention contain chiral centres and such compounds exist in the form of isomers (e.g. enantiomers). The invention includes all such isomers and any mixtures thereof including racemic mixtures.

Racemic forms can be resolved into the optical antipodes by known methods, for example, by separation of diastereomeric salts thereof with an optically active acid, and liberating the optically active amine compound by treatment with a base. Another method for resolving racemates into the optical antipodes is based upon chromatography on an optically active matrix. Racemic compounds of the present invention can thus be resolved into their optical antipodes, e.g., by fractional crystallisation of d- or l-(tartrates, mandelates, or camphorsulphonate) salts for example. The compounds of the present invention may also be resolved by the formation of diastereomeric derivatives.

Additional methods for the resolution of optical isomers, known to those skilled in the art, may be used. Such methods include those discussed by J. Jaques, A. Collet, and S. Wilen in “Enantiomers, Racemates, and Resolutions”, John Wiley and Sons, New York (1981).

Optically active compounds can also be prepared from optically active starting materials.

The compounds of the invention can be prepared by one of the following methods comprising:

a) reducing the carbonyl groups of a compound of formula

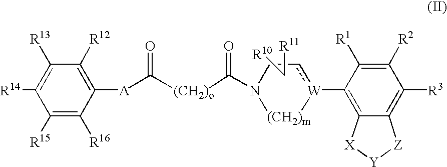

wherein o=0-8, m=2-3, and R¹-R³, R¹⁰, R¹¹, R¹²-R¹⁶, W, X, Y, Z, A, and the dotted line are as defined above;

b) reducing the carbonyl group of a compound of formula

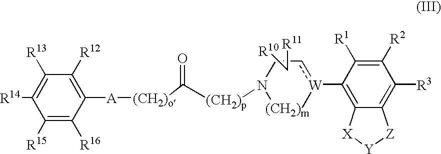

wherein p=0-4, o′=0-9, and R¹-R³, R¹⁰, R¹¹, R¹²-R¹⁶, W, X, Y, Z, A, m, and the dotted line are as defined above;

c) alkylating an amine of formula

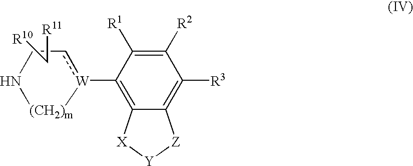

wherein R¹-R³, R¹⁰, R¹¹, W, X, Y, Z, m, and the dotted line are as defined above with a reagent of formula

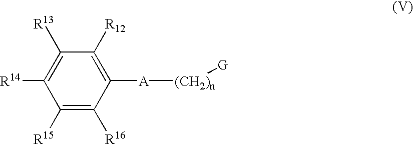

wherein R¹²-R¹⁶, A and n are as defined above and G is a suitable leaving group such as halogen, mesylate, or tosylate;

d) reductive alkylation of an amine of formula

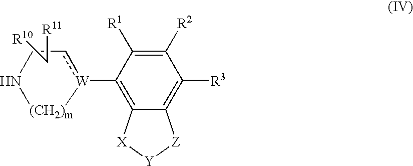

wherein R¹-R³, R¹⁰, R¹¹, W, X, Y, Z, m, and the dotted line are as defined above with a reagent of formula

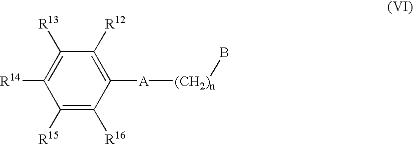

wherein R¹-R¹⁶, A and n are as defined above and B is either an aldehyde or a carboxylic acid derivative;

e) reducing the double bond of the unsaturated cyclic amines of formula

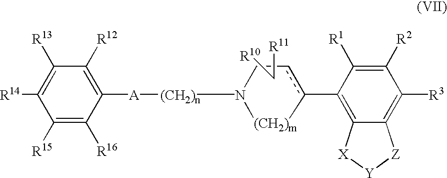

wherein R¹-R³, R¹⁰, R¹¹, R¹²-R¹⁶, A, X, Y, Z, m and n are as previously defined, in order to obtain the corresponding saturated derivatives;

f) treating a compound of general formula (I) wherein Y is —CR⁶═CR⁷—, or wherein X and Y together form a group —CR⁴═CR⁵—, or —CR⁴═CR⁵—CR⁶R⁷ with a reducing agent in order to reduce the double bond, thereby obtaining a corresponding reduced ring system;

g) reductive removal of one or more of the substituents R¹-R³ or R¹²-R¹⁶ in a compound of general formula (I) in which one or more of these substituents are selected from chloro, bromo, or iodo;

h) dialkylating an amine of formula

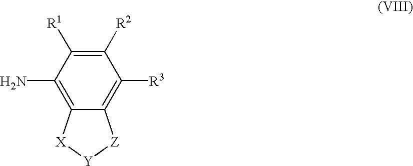

wherein R¹-R³, X, Y, Z, are as defined above with a reagent of formula

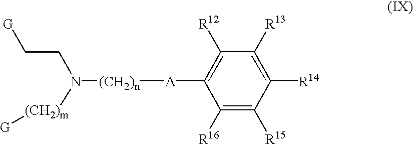

wherein R¹²-R¹⁶, A, m and n are as defined above and G is a suitable leaving group such as halogen, mesylate, or tosylate;

i) dialkylating an amine of formula

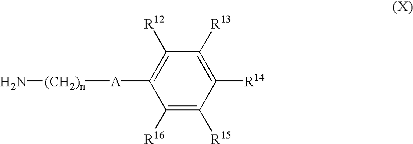

wherein R¹²-R¹⁶, A and n are as defined above, with a reagent of formula

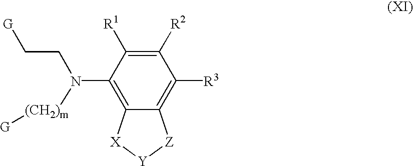

wherein R¹-R³, X, Y, Z, m, are as defined above and G is a suitable leaving group such as halogen, mesylate, or tosylate;

j) reduction of sulfones or sulfoxides of the formula

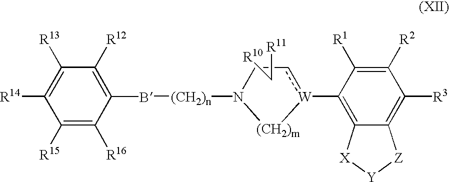

wherein R¹-R³, R¹⁰, R¹¹, R¹²-R¹⁶, W, X, Y, Z, m, n, and the dotted line are as defined above, and B′ is a sulfonyl or sulfinyl group;

k) alkylation of compounds of formula

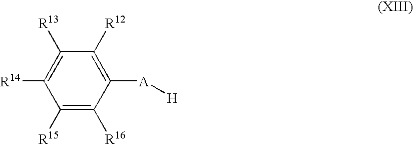

wherein R¹²-R¹⁶ and A are as defined above, with a reagent of formula

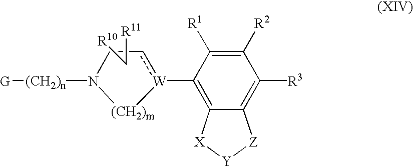

wherein R¹-R³, R¹⁰, R¹¹, W, X, Y, Z, m, n, and the dotted line are as defined above and G is a suitable leaving group such as halogen, mesylate, or tosylate;

whereupon the compounds of formula (I) are isolated as the free base or in the form of a pharmaceutically acceptable salt thereof.

The reduction according to methods a) and b) is preferably carried out in an inert organic solvent such as diethyl ether or tetrahydrofuran in the presence of lithium aluminium hydride at reflux temperature.

The alkylation according to method c) is conveniently performed in an inert organic solvent such as a suitably boiling alcohol or ketone, preferably in the presence of a base (potassium carbonate or triethylamine) at reflux temperature.

Arylpiperazine derivatives of formula (IV) are either commercially available or conveniently prepared from the corresponding arylamine according to the method described by Martin et al, J. Med. Chem., 1989, 32, 1052, or the method described by Kruse et al, Rec. Trav. Chim. Pays-Bas, 1988, 107, 303. The starting arylamines are either commercially available or are well-described in the literature.

Aryltetrahydropyridine derivatives of formula (IV) are known from literature, cf. U.S. Pat. No. 2,891,066; McElvain et al, J. Amer. Chem. Soc. 1959, 72, 3134. Conveniently, the corresponding arylbromide is lithiated with BuLi followed by addition of 1-benzyl-4-piperidone. Subsequent treatment with acid gives the N-benzyl-aryltetrahydropyridine. The benzyl group can be removed by catalytic hydrogenation or by treatment with e.g. ethyl chloroformate to give the corresponding ethyl carbamate followed by acidic or alkaline hydrolysis. The starting arylbromides are either commercially available or well-described in the literature.

Reagents of formula (V) are either commercially available or can be prepared by literature methods, e.g. from the corresponding carboxylic acid derivative by reduction to the 2-hydroxyethyl derivative and conversion of the hydroxy group to the group G by conventional methods, or from the corresponding dihalo alkyl or1-halo alkohol.

The reductive alkylation according to method d) is performed by standard literature methods. The reaction can be performed in two steps, i.e. coupling of (IV) and the reagent of formula (VI) by standard methods via the carboxylic acid chloride or by use of coupling reagents such as e.g. dicyclohexylcarbodiimide followed by reduction of the resulting amide with lithium aluminium hydride. The reaction can also be performed by a standard one-pot procedure. Carboxylic acids or aldehydes of formula (VI) are either commercially available or described in the literature.

Reduction of the double bonds according to methods e) and f) is most conveniently perfomed by hydrogenation in an alcohol in the presence of a noble metal catalyst, such as e.g. platinum or palladium.

The removal of halogen substituents according to method g) is conveniently performed by catalytic hydrogenation in an alcohol in the presence of a palladium catalyst or by treatment with ammonium formate in an alcohol at elevated temperatures in the presence of a palladium catalyst.

The dialkylation of amines according to methods h) and i) is most conveniently performed at elevated temperatures in an inert solvent such as e.g. chlorobenzene, toluene, N-methylpyrrolidone, dimethylformamide, or acetonitrile. The reaction might be performed in the presence of base such as e.g. potassium carbonate or triethylamine. Starting materials for processes h) and i) are commercially available or can be prepared from commercially available materials using conventional methods.

The N-alkylation according to method i) is performed in an inert solvent such as e.g. an alcohol or ketone at elevated temperatures in the presence of base, e.g. potassium carbonate or triethylamine at reflux temperature. Alternatively, a phase-transfer reagent can be used.

Reduction of sulfones and sulfoxides according to method j) can performed using several commercially available reagents as titaniumtetrachloride and sodiumborohydride at room temperature (S. Kano et al. Synthesis 1980, 9, 695-697).

Alkylation of commercially available compounds corresponding to formula (XIII) using method k) is conveniently performed using a alkylating reagent with the appropriate leaving group (eg. mesylate, halide) using a base (eg. potassium carbonate or similar) in a polar aprotic solvent (eg. methyl isobutylketone, dimethylformamide).

Arylpiperazines used as described in the examples are prepared from the corresponding arylamine according to the method described by Martin et al, J. Med. Chem. 32 (1989) 1052, or the method described by Kruse et al, Rec. Trav. Chim. Pays-Bas 107 (1988) 303. The starting arylamines are either commercially available or are described in the literature as follows:

The synthesis of 5-amino-1,4-benzodioxane is described by Dauksas et al, Zh. Org. Khim., 1967, 3, 1121. The corresponding chlorinated derivatives are made in a similar manner.

The synthesis of 7-amino-2,3-dihydrobenzofuran is described in U.S. Pat. No. 4,302,592.

The synthesis of 7-amino-benzofuran is described by Van Wijngaarden et al, J. Med. Chem., 1988,31, 1934.

The synthesis of 7-amino-benzo[b]thiophene is described by Boswell et al , J. Heterocycl. Chem., 1968, 5, 69.

7-amino-2,3-dimethylbenzofuran and the corresponding 5-chloro and 5-methyl derivatives are prepared according to Ger. Offen. DE 3526510.

4-Amino-benzothiopyran were prepared according to Eur. Pat. Appl. EP 79683.

8-Amino-6-chloro-2,2-dimethylebenzopyran was prepared by conventional nitration of 6-chloro-2,2-dimethylebenzopyran (prepared according to Bolzoni et al, Angew. Chem., 1978, 90, 727-) with subsequent reduction of the obtained 8-nitro derivative. In a similar manner 7-amino-5-chloro-3,3-dimethylbenzofuran was obtained from 5-chloro-3,3-dimethylbenzofuran (prepared according to Eur. Pat. Appl. EP 7719 800206). The corresponding dechloro derivatives were obtained by treatment with hydrogen gas in the presence of a noble metal catalyst according to standard procedures.

Aryl tetrahydropyridine derivatives are known from literature (cf. U.S. Pat. No. 2,891,066 or McElvain et al, J. Amer. Chem. Soc., 1959, 72, 3134). Most conveniently, the corresponding aryl bromide is lithiated with BuLi followed by addition of 1-benzyl-4-piperidone. Subsequent treatment with mineral acid or trifluoroacetic acid gives the N-benzyl-aryltetrahydropyridine. The benzyl group can be removes by catalytic hydrogenation or by treatment e.g. ethyl chloroformate to the corresponding ethyl carbamate followed by acidic or alkaline hydrolysis. The corresponding piperidine derivatives can be obtained by reductive removal of the double bond of the tetrahydropyridine ring. All these procedures are well-known to a person skilled in the art. The starting aryl bromides are well-described in the literature. In this manner 4-(1,4-benzodioxan-5-yl)-1,2,3,6-tetrahydropyridine, 4-(2,3-dihydro-2,2-dimethylbenzofuran-7-yl)-1,2,3,6-tetrahydropyridine, 4-(2,3-dihydrobenzofuran-7-yl)-1,2,3,6-tetrahydropyridine, 4-(benzofuran-7-yl)-1,2,3,6-tetrahydropyridine, and the corresponding piperidines were obtained.

The following examples will illustrate the invention further. They are, however, not to be construed as limiting.

### EXAMPLES

Melting points were determined on a Büchi SMP-20 apparatus and are uncorrected. Analytical LC-MS data were obtained on a PE Sciex API 150EX instrument equipped with IonSpray source (method D) or heated nebulizer (APCI, methods A and B) and Shimadzu LC-8A/SLC-10A LC system. The LC conditions [30×4.6 mm YMC ODS-A with 3.5 μm particle size] were linear gradient elution with water/acetonitrile/trifluoroacetic acid (90:10:0.05) to water/acetonitrile/trifluoroacetic acid (10:90:0.03) in 4 min at 2 mL/min. Purity was determined by integration of the UV trace (254 nm). The retention times R. are expressed in minutes.

Mass spectra were obtained by an alternating scan method to give molecular weight information. The molecular ion, MH+, was obtained at low orifice voltage (5-20V) and fragmentation at high orifice voltage (100V).

Preparative LC-MS-separation was performed on the same instrument. The LC conditions (50×20 mm YMC ODS-A with 5 μm particle size) were linear gradient elution with water/acetonitrile/trifluoroacetic acid (80:20:0.05) to water/acetonitrile/trifluoroacetic acid (10:90:0.03) in 7 min at 22.7 mL/min. Fraction collection was performed by split-flow MS detection.

¹H NMR spectra were recorded at 500.13 MHz on a Bruker Avance DRX500 instrument or at 250.13 MHz on a Bruker AC 250 instrument. Deuterated chloroform (99.8%D) or dimethyl sulfoxide (99.9%D) were used as solvents. TMS was used as internal reference standard. Chemical shift values are expressed in ppm-values. The following abbreviations are used for multiplicity of NMR signals: s=singlet, d=doublet, t=triplet, q-quartet, qui=quintet, h=heptet, dd=double doublet, dt=double triplet, dq=double quartet, tt=triplet of triplets, m=multiplet, b=broad singlet. NMR signals corresponding to acidic protons are generally omitted. Content of water in crystalline compounds was determined by Karl Fischer titration. Standard workup procedures refer to extraction with the indicated organic solvent from proper aqueous solutions, drying of combined organic extracts (anhydrous MgSO₄ or Na₂SO₄), filtering and evaporation of the solvent in vacuo. For column chromatography silica gel of type Kieselgel 60, 230-400 mesh ASTM was used. For ion-exchange chromatography (SCX, 1 g, Varian Mega Bond Elut®, Chrompack cat. no. 220776). Prior use the SCX-columns were pre-conditioned with 10% solution of acetic acid in methanol (3 mL).

### Example 1

1a. 1-[3-(2-Chloro-phenoxy)-propyl]-4-(2,3-dihydro-benzo[1,4]dioxin-5-yl)-piperazine, Oxalate.

A solution of 2-chlorophenol (5 g) in tetrahydrofuran (25 mL) was added dropwise to a slurry of sodiumhydride (47 mmol) in tetrahydrofuran (50 mL) at room temperature. The mixture was stirred for 30 min. The reaction mixture was then warmed to reflux whereafter 2-bromopropanol (3.5 mL) in tetrahydrofuran (25 mL) was added over 5 min. The mixture was refluxed over night, one more equivalent of 3-bromopropanol was added and the mixture was refluxed for 12 hrs more. The mixture was cooled, brine and ethylacetate added, and washed using standard procedure. The combined organic phases were dried and evaporated. The crude 3-(2-chlorophenoxy)-1-propanol was dissolved in acetonitrile (500 mL) and carbon tetrabromide (38.7 g) was added. To the cooled (0° C.) mixture triphenylphosphine (25.5 g) was added portionwise over 30 min. The reaction was allowed to react at room temperature for 3 hrs, then evaporated to give an oily product. The crude product was purified using silica gel flash chromatography (heptane: ethylacetate: triethylamine/70:15:5) to give 3-(2-chlorophenoxy)-1-propyl bromide (10.7 g). A mixture of 1-(1,4-benzodioxan-5-yl)piperazine (0.84 g), potassium carbonate (1.6 g), potassium iodide (cat.) and 3-(2-chlorophenoxy]-1-propyl bromide (1.0 g) in methyl isobutylketone/dimethylformamide (1/1, 100 mL) was heated to 120° C. When TLC indicated the reaction to be completed (24 hrs) the mixture was cooled, filtered and concentrated. The crude material was dissolved in ethyl acetate and washed using standard procedure, followed by drying, filtration and evaporation. The crude materials were purified using silica gel flash chromatography (heptane: ethylacetate: triethylamine/55:43:2). The resulting oil was dissolved in acetone followed by addition of oxalic acid. Filtration gave the title compound as pure crystalline material (0.6 g). Mp 163-166° C. ¹H NMR: 2.15 (m, 2H); 3.00-3.20 (m, 10H); 4.15 (t, 2H); 4.20 (m, 4H); 6.50 (d, 1H); 6.55 (d, 1H); 6.75 (dd, 1H); 6.95 (d, 1H); 7.15 (d, 1H); 7.30 (dd, 1H); 7.40 (d, 1H). MS: m/z: 389 (MH+), 218, 150. Anal. Calcd for C₂₁H₂₅ClN₂O₃: C, 57.67; H, 5.69; N, 5.85. Found C, 57.71; H, 5.74; N, 5.77.

The following compounds were prepared analogously:

1b. 1-[3-(2,6-Dichloro-phenoxy)-propyl]-4-(2,3-dihydro-benzo[1,4]dioxin-5-yl)-piperazine, oxalate. Mp 179-181° C. ¹H NMR: 2.15 (m, 2H); 3.00-3.20 (m, 10H); 4.05 (t, 2H); 4.20 (m, 4H); 6.50 (d, 1H); 6.55 (d, 1H); 6.75 (dd, 1H); 7.20 (dd, 1H); 7.50 (d, 2H). MS: m/z: 423 (MH+), 247, 178. Anal. Calcd for C₂₁H₂₄Cl₂N₂O₃: C, 53.80; H, 5.1 1; N, 5.46. Found C, 53.73; H, 5.01; N, 5.40.

1c. 1-(2,3-Dihydro-benzo[1,4]dioxin-5-yl)-4-[3-(2,4,6-trifluoro-phenoxy)-propyl]-piperazine, dihydrochloride. Mp 210-220° C. ¹H NMR: 2.10 (m, 2H); 3.05-3.25 (m, 10H); 3.80 (s, 3H); 4.00 (t, 2H); 4.25 (m, 4H); 6.50 (d, 1H); 6.55 (d, 1H); 6.65-6.80 (m, 2H); 6.85-7.00 (m, 2H); 11.25 (b, 1H). MS: m/z: 409 (MH+), 232, 150. Anal. Calcd for C₂₁H₂₃F₃N₂O₃: C, 52.39; H, 5.25; N, 5.82. Found C, 52.63; H, 5.40; N, 5.71.

1d. 1-(2,3-Dihydro-benzo[1,4]dioxin-5-yl)-4-[3-(4-fluoro-2-methoxy-phenoxy)-propyl]-piperazine, oxalate. Mp 141-142° C. ¹H NMR: 2.10 (m, 2H); 3.05-3.25 (m, 10H); 3.80 (s, 3H); 4.00 (t, 2H); 4.25 (m, 4H); 6.50 (d, 1H); 6.55 (d, 1H); 6.65-6.80 (m, 2H); 6.85-7.00 (m, 2H). MS: m/z: 403 (MH+), 164. Anal. Calcd for C₂₂H₂₇FN₂O₄: C, 58.52; H, 5.95; N, 5.69. Found C, 58.53; H, 6.24; N, 5.22.

1e. 1-(2,3-Dihydro-benzo[1,4]dioxin-5-yl)-4-[3-(4-fluoro-2-methyl-phenoxy)-propyl]-piperazine, oxalate. Mp 139-150° C. ¹H NMR: 2.05-2.15 (m, 2H); 2.15 (s, 3H); 3.05-3.20 (m, 10H); 4.00 (t, 2H); 4.20-4.25 (m, 4H); 6.50 (d, 1H); 6.55 (d, 1H); 6.75 (dd, 1H); 6.95 (m, 2H); 7.00 (m, 1H). MS: m/z: 387 (MH+), 218, 164. Anal. Calcd for C₂₂H₂₇FN₂O₃: C, 59.92; H, 6.19; N, 5.82. Found C, 59.82; H, 5.32; N, 5.49.

### Example 2

2a, 1-[3-(4-Chloro-phenoxy)-propyl]-4-(2,3-dihydro-benzo[1,4]dioxin-5-yl)-piperazine. A solution of 4-chlorophenol (5 g) in dimethylformamide (50 mL) was added dropwise to a slurry of sodiumhydride (60%, 1.7 g) in dimethylformamide (50 mL) at room temperature over 15 min. The mixture was stirred for 30 min. The reaction mixture was then slowly (10 min) added to a solution of 1,3-dibromopropane (78.5 g) in dimethylformamide (25 mL) at roomtemperature. The final mixture was stirred for further 60 min at 70° C. The reaction to was quenched by addition of sufficient amounts of water to destroy excess sodiumhydride, acidified using etheral hydrogen chloride followed by evaporation. The crude oil was purified using silicagel flash chromatography, (heptane: ethylacetate: triethylamine/ 95:2.5:2.5) to give 3-(4-chlorophenoxy)-1-propyl bromide (4.5 g).

A mixture of 1-(1,4-benzodioxan-5-yl)piperazine (1.0 g), potassium carbonate (1.9 g), potassium iodide (cat.) and 3-(4-chlorophenoxy)-1-propyl bromide (1.13 g) in methyl isobutylketone/dimethylformamide (1/1, 100 mL) was heated to 120° C. When TLC indicated the reaction to be completed (24 hrs) the mixture was cooled, filtered and evaporated. The crude material was dissolved in ethylacetate and washed using standard procedure, followed by drying, filtration and concentration. The crude material was purified using silica gel chromatography (heptane: ethylacetate: ethanol: triethylamine/85:5:25:5). The collected oil was crystallized from ethanol. Filtration gave the title compound as pure crystalline material (0.64 g). Mp 116-119° C. ¹H NMR: 1.90 (q, 2H); 2.40-2.60 (m, 6H); 2.90-3.00 (m, 4H); 4.00 (t, 2H); 4.20 (m, 4H); 6.45 (m, 2H); 6.70 (t, 1H); 6.95 (d, 2H); 7.30 (d, 2H). MS: m/z: 389 (MH+), 178. Anal. Calcd for C₂₁H₂₅ClN₃N₂O₃: C, 64.86; H, 6.48; N, 7.20. Found C, 64.59; H, 6.49; N, 7.23.

The following compounds were prepared analogously:

2b, 1-(2,3-Dihydro-benzo[1,4]dioxin-5-yl)-4-[3-(4-trifluoromethyl-phenoxy)-propyl]-piperazine, oxalate. Mp 148-150° C. ¹H NMR: 2.10 (m, 2H); 3.00-3.25 (m, 10H); 4.15 (t, 2H); 4.25 (m, 4H); 6.45-6.55 (m, 2H); 6.75 (t, 1H); 7.15 (d, 2H); 7.60 (d, 2H). MS: m/z: 423 (MH+), 178. Anal. Calcd for C₂₂H₂₅F₃N₂O₃: C, 56.25; H, 5.31; N, 5.47. Found C, 56.10; H, 5.34; N, 5.51.

2c. 1-(2,3-Dihydro-benzo[1,4]dioxin-5-yl)-4-[3-(2-fluoro-phenoxy)-propyl]-piperazine, oxalate. Mp 167-169° C. ¹H NMR: 2.10 (m, 2H); 3.00-3.20 (m, 10H); 4.15 (t, 2H); 4.20 (m, 4H); 6.45-6.55 (m, 2H); 6.75 (t, 1H); 6.95 (m, 1H); 7.10-7.25 (m, 3H). MS: m/z: 373 (MH+), 178, 122. Anal. Calcd for C₂₂H₂₅FN₂O₃: C, 59.73; H, 5.88; N, 6.06. Found C, 59.15; H, 5.99; N, 6.04.

2d. 2-{3-[4-(2,3-Dihydro-benzo[1,4]dioxin-5-yl)-piperazin-1-yl]-propoxy}-benzonitrile, oxalate. Mp 130 (amorphous) C. ¹H NMR: 2.15 (m, 2H); 3.00-3.20 (m, 10H); 4.20-4.30 (m, 6H); 6.50 (d, 1H); 6.55 (d, 1H); 6.75 (t, 1H); 7.10 (t, 1H); 7.25 (d, 1H); 7.65-7.75 (m, 2H). MS: m/z: 380 (MH+), 178. Anal. Calcd for C₂₂H₂₅N₃O₃: C, 61.40; H, 5.80; N, 8.95. Found C, 59.97; H, 6.02; N, 8.72.

2e. 1-Benzo[b]thiophen-7-yl-4-[3-(2-chloro-4-fluoro-phenylsulfanyl)-propyl]-piperazine, hydrochloride. Mp 216-219° C. ¹H NMR: 2.06-2.17 (m, 2H); 3.10-3.18 (t, 2H); 3.21-3.35 (m, 6H); 3.58-3.69 (d, 4H); 7.02 (d, 1H); 7.27 (t, 1H); 7.38 (t, 1H); 7.48 (d, 1H); 7,52-7.60 (m, 2H); 7,62 (d, 1H); 7.77 (d, 1H); 11.0 (s, 11H). MS: m/z: 421 (MH+), 299, 176. Anal. Calcd for C₂₁H₂₂ClFN₂S₂: C, 55.13; H, 5.08; N, 6.12. Found C, 55.06; H, 5.09; N, 6.15.

2f. 1-Benzo[b]thiophen-7-yl-4-[4-(2-chloro-4-fluoro-phenoxy)-butyl]-piperazine, hydrochloride. Mp 193-195° C. ¹H NMR: 1.80-1.88 (m, 2H); 1.95-2.06 (m, 2H); 3.18-3.42 (m, 6H); 4.05-4.14 (m, 2H); 7.05 (d, 1H); 7.20 (t, 1H); 7.43 (m, 3H); 7.63 (d, 1H); 7.77 (d, 1H); 11.30(s, 1H). MS: m/z: 419 (MH+), 216, 134. Anal. Calcd for C₂₂H₂₄ClFN₂OS: C, 58.01; H, 5.54; N, 6.15. Found C, 57.89; H, 5.54; N, 6.19.

### Example 3

3a, 1-[2-(3,4-Dichloro-phenylsulfanyl)-ethyl]-4-(2,3-dihydro-benzof[, 4]dioxin-5-yl)-piperazine, oxalate. A solution of chloroacetyl chloride (0.72 g) in dry tetrahydrofuran (5 mL) was added dropwise to a mixture of 1-(1,4-benzodioxan-5-yl)piperazine (1.28 g) and potassium carbonate (2.4 g) in dry tetrahydrofuran at room temperature. The reaction was allowed to stir for 30 min. and 3,4-dichlorothiophenol (1.25 g) was added followed by addition of potassium tert-butoxide (1.49 g). The mixture was stirred 30 min at room temperature and 30 min at reflux, whereafter it was cooled and concentrated. The crude mixture was washed using standard procedure (ethylacetate/brine), dried and evaporated to give 1-[1,4-benzodioxan-5-yl]-4-[3,4-dichlorophenylthiomethylcarbonyl]piperazine (2.54 g).

Aluminium trichloride (0.4 g) in cold tetrahydrofuran (10 mL) was added dropwise to a suspension of lithium aluminium hydride (0.4 g) in tetrahydrofuran (20 mL) at 0° C. The mixture was stirred for 15 min and then allowed to warm to approx. 10° C., whereafter a solution of the intermediate amide, prepared above, in tetrahydrofuran (20 mL) was added. The reaction was complete after 1 h and concentrated sodium hydroxide (2 mL) was added, dropwise. Drying agent was added followed by filtration and evaporation to give the crude target base (1.94 g). Purification using silica gel flash chromatography gave the pure base. Addition of oxalic acid in acetone followed by filtration gave the title compound as pure white crystalline material (1.26 g). Mp 159-161° C. ¹H NMR: 2.9-3,05 (s, 6H); 3.05-3.15(s, 4H); 3.25-3.40 (t, 2H); 4.15-4.30 (m, 4H); 4.70-6.40 (b, 1H); 6.45-6.50 (d, 1H); 6.50-6.55 (d, 1H); 6.70-6.80 (t, 1H); 7.30-7.40 (d, 1H); 7.55-7.60 (d, 1R); 7.65-7.67 (s, 1H). MS m/z: 425 (MH+), 177. Anal. Calcd for C₂₀H₂₂Cl₂N₂O₂S: C, 51.26; H, 4.70; N, 5.44. Found C, 51.41; H, 4.86; N, 5.44.

The following compounds were prepared analogously:

3b. 1-(2,3-Dihydro-benzo[1,4]dioxin-5-yl)-4-[2-(4-fluoro-phenylsulfanyl)-ethyl]-piperazine, oxalate. Mp 200-202° C. ¹H NMR: 2.90-3.10 (m, 6H); 3.15-3.30 (s, 4H); 3.30-3.40 (t, 2H); 3.60-4.50 (b, 1H); 6.35-6.40(s, 1H); 6.45-6.50 (d, 1H); 6.95-7.00 (t, 1H); 7.05-7.10 (d, 1H); 7.15-7.20 (s, 1H); 7.25-7.30 (s, 1H); 7.35-7.40 (d, 1H); 7.55-7.60 (d, 1H). MS m/z: 375 (MH+), 177. Anal. Calcd for C₂₀H₂₃FN₂O₂S: C, 56.88; H, 5.44; N, 6.03. Found C, 56.88; H, 5.55; N, 5.96.

3c. 1-[2-(Bromo-trifluoromethyl-phenylsulfanyl)-ethyl]-4-(2,3-dihydro-benzo[1,4]dioxin-5-yl)-piperazine, oxalate. Mp 196-197° C. ¹H NMR: 2.65-2.85 (m, 4H); 2.85-2.95 (m, 2H); 2.95-3.15 (s, 4H); 3.15-3.35 (m, 2H); 4.15-4.40 (dd, 4H); 6.40-6.55 (m, 2H); 6.70 (t, 1H); 7,57 (d, 1H); 7.73 (d, 1H); 7.95 (s, 1H). MS m/z: 504 (MH+), 214. Anal. Calcd for C₂₀H₂₂BrF₃N₂O₂S: C, 45.5 1; H, 4.24; N, 4.62. Found C, 46.00; H, 4.25; N, 4.58.

3d. 1-[2-(2,6-Dichloro-phenylsulfanyl)-ethyl]-4-(2,3-dihydro-benzo[1,4]dioxin-5-yl)-piperazine, oxalate. Mp 188-191° C. (decomposes). ¹H NMR: 2,85-3,0(m, 6H); 3.00-3.15 (s, 4H); 3.20 (t, 2H); 4.15-4.25 (m, 4H); 5.00-6.00 (b, 1H); 6.45(d, 1H); 6.50 (d, 1H); 6.70(t, 1H); 7.40(t, 1H); 7.60(d, 2H). MS m/z. 425 (MH+), 170. Anal. Calcd for C₂₀H₂₂Cl₂N₂O₂S: C, 51.27; H, 4.69; N, 5.44. Found C, 51.17; H, 4.81; N, 5.46.

### Example 4

4a 1-(2,3-Dihydro-benzo[1,4]dioxin-5-yl)-4-(3-phenylsulfanyl-propyl)-piperazine, dihydrochloride hydrate. To a stirred solution of concentrated sodiumhydroxide (100 mL), dichloromethane (900 mL) and water (600 mL), was added thiophenol (56 g), 3-bromopropan-1-ol (111 g) and tetrabutylammonium sulphate (12 g). The mixture was refluxed for 42 h, slowly cooled, followed by washing using dichloromethane/hydrochloric acid and water, drying and evaporation to give crude 3-phenylthiopropan-1-ol which was purified by distillation (35 g, bp 102-15° C./0.15 mmHg. A portion (10 g), was dissolved in dichloromethane (100 mL) and triethylamine (8.6 g) was added, followed by dropwise addition of a dichloromethane (100 mL) solution of methanesulfonic acid chloride (9.3 g) at 2° C. The reaction was allowed to proceed at this temperature for 90 min and and at 10° C. for same amount of time. The reaction was then washed using dichloromethane and diluted sodiumcarbonate solution, dried and evaporated to give the crude mesylate (14.9 g). The mesylate (3.1 g) was directly treated with 1-(1,4-benzodioxan-5-yl)piperazine, dihydrochloride (3.22 g) and potassium carbonate (9.15 g) in methyl isobutylketone (120 mL). The reaction was refluxed for 48 h, cooled, evaporated then washed using standard procedure. Purification using silica gel flash chromatography gave the target base (0.56 g), which was crystallized as the hydrochloride by addition of etheral hydrogen chloride. Filtration yielded the title compound (0.50 g). Mp 185-206° C. ¹H NMR: 2.00-2.16 (m, 2H); 3.03-3.30 (m, 8H); 3.34-3.55 (m, 4H); 4.18-4.25 (s, 4H); 5.80 (s, 4H); 6.48-6.61 (m, 2H); 6.73 (t, 1H); 7.14-7.25 (m, 1H), 7.28-7.32 (m, 4H); 11.48 (s, 1H). MS m/z: 371 (MH+). Anal. Calcd for C₂₁H₂₆N₂O₂S: C, 54.73; H, 6.56; N, 6.08. Found C, 55.37; H, 6.65; N, 6.27.

### Example 5

5aa. 1-[3-(2-Bromo-4-fluoro-phenoxy)-propyl]-4-(2,3-dihydro-benzo[1,4]dioxin-5-yl)-piperazine.

A solution of 2-bromo-4-fluoro-phenol (3.0 g) in tetrahydrofuran (50 ml) was added dropwise at room temperature to a suspension of sodium hydride (38.4 mmol) in ethanol (50 ml). The mixture was stirred for an additional 30 min after the generation of hydrogen stopped. The solution was then slowly dropped (0.3 mL/min) to a solution of 1,3-dibromopropane (159 g) in ethanol (300 mL) at 75° C. and stirred for 16 h. The mixture was evaporated from the solvents and the residue was extracted with ethyl acetate. The solution was washed with water and brine, dried, filtered and concentrated. The excess 1,3-dibromopropane was removed in vacuo (60° C., 0.01 mbar) and the oily residue was purified by silica gel flash chromatography (eluent:heptane) to yield 3-(2-bromo-4-fluorophenoxy)-1-propyl bromide (2.9 g, 60%) as a colorless oily liquid.

Cesium carbonate (108 mg) was added to a solution of 3-(2-bromo-4-fluorophenoxy)-1-propyl bromide (46 mg) and 1-(1,4-benzodioxan-5-yl)piperazine (26 mg) in acetonitril (2 mL). The mixture was stirred at 70° C. for 16 h. After 12 h isocyanomethyl polystyrene (75 mg) was added and the mixture was slowly cooled to room temperature. The resin was filtered and washed with methanol (1×1 mL) and dichloromethane (1×1 mL). The combined liquid phases were evaporated from volatile solvents to yield a dark brown oil. The crude product was dissolved in ethyl acetate (3 mL) and loaded on a pre-conditioned ion exchange column. The column was washed with methanol (4 mL) and acetonitrile (4 mL), followed by elution of the product with 4 N solution of ammonia in methanol (4.5 mL). After evaporation of volatile solvents the product was purified by preparative reversed phase HPLC chromatography. The resulting solution was again loaded on a pre-conditioned ion exchange column. As described above the column was washed with methanol (4 mL) and acetonitrile (4 mL), followed by elution of the product with 4 N solution of ammonia in methanol (4.5 mL). Evaporation of the volatile solvents afforded the title compound as a yellow oil (34 mg). LC/MS (m/z) 451 (MH+), Rt=6.0 (method A), purity: 95.6%.

The following compounds where prepared analogously:

(Method A)

5ab. 1-[4-(2,6-Dichloro-phenylsulfanyl)-butyl]-4-(2,3-dihydro-benzo[1,4]dioxin-5-yl)-piperazine.

LC/MS (m/z) 453 (MH+), Rt=2.52(method A), purity 96.1%.

5ac. 1-[3-(2-Chloro-4-fluoro-phenylsulfanyl)-propyl]-4-(2,3-dihydro-benzo[1,4]dioxin-5-yl)-piperazine.

LC/MS (m/z) 424 (MH+), Rt=5.75 (method A), purity 91.8%.

5ad. 1-Benzo[b]thiophen-7-yl-4-[3-(2-chloro-4-fluoro-phenylsulfanyl)-propyl]-piperazine.

LC/MS (m/z) 421 (MH+), Rt=6.40 (method A), purity 73.2%.

5ae. 1-Benzo[b]thiophen-7-yl-4-[3-(2,6-dichloro-phenylsulfanyl)-propyl]-piperazine.

LC/MS (m/z) 437 (MH+), Rt=6.39 (method A), purity 84.1%.

5af. 1-Benzo[b]thiophen-7-yl-4-[4-(2,6-dichloro-phenylsulfanyl)-butyl]-piperazine.

LC/MS (m/z) 451 (MH+), Rt=6.64 (method A), purity 87.6%.

5ag. 1-[4-(3-Chloro-2-methoxy-phenylsulfanyl)-butyl]-4-(2,3-dihydro-henzo[1,4]dioxin-5-yl)-piperazine.

LC/MS (m/z) 449 (MH+), Rt=5.91 (method A), purity 90.8%.

5ah. 1-Benzo[b]thiophen-7-yl-4-[4-(3-chloro-2-methoxy-phenylsulfanyl)-butyl]-piperazine.

LC/MS (m/z) 447 (MH+), Rt=6.54 (method A), purity 73.8%.

5ai. 1-Benzo[b]thiophen-7-yl-4-[3-(2-chloro-4-fluoro-phenylsulfanyl)-propyl]-piperazine.

LC/MS (m/z) 422 (MH+), Rt=6.32 (method A), purity 94.2%.

5aj. 1-[3-(2,6-Dibromo-4-fluoro-phenoxy)-propyl]-4-(2,3-dihydro-benzo[1,4]dioxin-5-yl)-piperazine.

LC/MS (m/z) 531 (MH+), Rt=5.87 (method A), purity 96.4%.

5ak. 1-Benzo[b]thiophen-7-yl-4-[3-(2,6-dibromo-4-fluoro-phenoxy)-propyl]-piperazine.

LC/MS (m/z) 529 (MH+), Rt=6.98 (method A), purity 87.7%.

5al. 4-{3-[4-(2,3-Dihydro-benzo[1,4]dioxin-5-yl)-piperazin-1-yl]-propoxy}-3,5-diiodo-benzonitrile.

LC/MS (m/z) 632 (MH+), Rt=5.85 (method A), purity 86.0%.

5am. 3,5-Di-tert-butyl-4-{3-[4-(2,3-dihydro-benzo[1,4]dioxin-5-yl)-piperazin-1-yl]-propoxy}-benzonitrile.

LC/MS (m/z) 492 (MH+), Rt=6.74 (method A), purity 83.6%.

5an. 1-[3-(2,6-Dichloro-4-methanesulfonyl-phenoxy)-propyl]-4-(2,3-dihydro-benzo[1,4]dioxin-5-yl)-piperazine.

LC/MS (m/z) 503 (MH+), Rt=5.06 (method A), purity 93.6%.

5ao. 1-Benzo[b]thiophen-7-yl-4-[3-(2,6-dichloro-4-methanesulfonyl-phenoxy)-propyl]-piperazine.

LC/MS (m/z) 499 (MH+), Rt=5.82 (method A), purity 80.1%.

5ap. 1-[3-(Bromo-trifluoromethyl-phenylsulfanyl)-propyl]-4-(2,3-dihydro-benzo[1,4]dioxin-5-yl)-piperazine.

LC/MS (m/z) 519 (MH+), Rt=6.27 (method A), purity 86.5%.

5aq. 1-Benzo[b]thiophen-7-yl-4-[3-(bromo-trifluoromethyl-phenylsulfanyl)-propyl]-piperazine.

LC/MS (m/z) 517 (MH+), Rt=6.86 (method A), purity 73.7%.

5ar. 1-Benzo[b]thiophen-7-yl-4-[4-(2-chloro-6-methyl-phenylsulfanyl)-butyl]-piperazine.

LC/MS (m/z) 431 (MH+), Rt=6.66 (method A), purity 87.4%.

5as. 1-Benzo[b]thiophen-7-yl-4-[4-(2-chloro-4-fluoro-phenylsulfanyl)-butyl]-piperazine.

LC/MS (m/z) 435 (MH+), Rt=6.94 (method A), purity 83.0%.

5at. 1-[3-(2,6-Dichloro-4-fluoro-phenoxy)-propyl]-4-(2,3-dihydro-benzo[1,4]dioxin-5-yl)-piperazine.

LC/MS (m/z) 441 (MH+), Rt=5.80 (method A), purity 96.8%.

5au. 1-Benzo[b]thiophen-7-yl-4-[3-(2,6-dichloro-4-fluoro-phenoxy)-propyl]-piperazine.

LC/MS (m/z) 439 (MH+), Rt=6.49 (method A), purity 93.6%.

5av. 1-[4-(2-Chloro-6-methyl-phenylsulfanyl)-butyl]-4-(2,3-dihydro-benzo[1,4]dioxin-5-yl)-piperazine.

LC/MS (m/z) 433 (MH+), Rt=6.14 (method A), purity 96.6%.

5aw. 1-[3-(2,6-Dichloro-phenylsulfanyl)-propyl]-4-(2,3-dihydro-benzo[1,4]dioxin-5-yl)-piperazine.

LC/MS (m/z) 439 (MH+), Rt=5.89 (method A), purity 93.0%.

5ax. 1-(5-Chloro-2,2-dimethyl-2,3-dihydro-benzofuran-7-yl)-4-[4-(2-chloro-6-methyl-phenylsulfanyl)-butyl]-piperazine.

LC/MS (m/z) 479 (MH+), Rt=7.38 (method A), purity 91.3%.

5ay. 1-(5-Chloro-3,3-dimethyl-2,3-dihydro-benzofuran-7-yl)-4-[4-(2-chloro-6-methyl-phenylsulfanyl)-butyl]-piperazine.

LC/MS (m/z) 479 (MH+), Rt=7.38 (method A), purity 93.1%.

5az. 1-(5-Chloro-2,2-dimethyl-2,3-dihydro-benzofuran-7-yl)-4-[3-(2,6-dichloro-phenylsulfanyl)-propyl]-piperazine.

LC/MS (m/z) 488 (MH+), Rt=6.92 (method A), purity 93.1%.

5ba. 1-(5-Chloro-3,3-dimethyl-2,3-dihydro-benzofuran-7-yl)-4-[3-(2,6-dichloro-phenylsulfanyl)-propyl]-piperazine.

LC/MS (m/z) 488 (MH+), Rt=6.91 (method A), purity 88.7%.

5bb. 1-(5-Chloro-3,3-dimethyl-2,3-dihydro-benzofuran-7-yl)-4-[3-(2-chloro-4-fluoro-phenylsulfanyl)-propyl]-piperazine.

LC/MS (m/z) 469 (MH+), Rt=6.84 (method A), purity 88.8%.

5bc. 1-Benzo[b]thiophen-7-yl-4-[4-(2-chloro-4-fluoro-phenoxy)-butyl]-piperazine.

LC/MS (m/z) 419 (MH+), Rt=6.44 (method A), purity 98.5%.

5bd. 1-(5-Chloro-2,2-dimethyl-2,3-dihydro-benzofuran-7-yl)-4-[4-(2-chloro-4-fluoro-phenoxy)-butyl]-piperazine.

LC/MS (m/z) 467 (MH+), Rt=6.91 (method A), purity 94.2%.

5be. 1-[4-(2-Bromo-4-fluoro-phenoxy)-butyl]-4-(2,3-dihydro-benzo[1,4]dioxin-5-yl)-piperazine.

LC/MS (m/z) 467 (MH+), Rt=5.94 (method A), purity 99.3%.

5bf. 1-Benzo[b]thiophen-7-yl-4-[4-(2-bromo-4-fluoro-phenoxy)-butyl]-piperazine.

LC/MS (m/z) 465 (MH+), Rt=6.57 (method A), purity 99.7%.

5bg. 1-[4-(2-Bromo-4-fluoro-phenoxy)-butyl]-4-(5-chloro-2,2-dimethyl-2,3-dihydro-benzofuran-7-yl)-piperazine.

LC/MS (m/z) 514 (MH+), Rt=7.02 (method A), purity 99.2%.

5bh. 1-(5-Chloro-2,2-dimethyl-2,3-dihydro-benzofuran-7-yl)-4-[3-(2,6-dichloro-4-methanesulfonyl-phenoxy)-propyl]-piperazine.

LC/MS (m/z) 549 (MH+), Rt=6.34 (method A), purity 88.6%.

5bi. 1-(5-Chloro-3,3-dimethyl-2,3-dihydro-benzofuran-7-yl)-4-[3-(2,6-dichloro-4-methanesulfonyl-phenoxy)-propyl]-piperazine.

LC/MS (m/z) 549 (MH+), Rt=6.43 (method A), purity 84.0%.

5bj. 1-(5-Chloro-3,3-dimethyl-2,3-dihydro-benzofuran-7-yl)-4-[4-(3-chloro-2-methoxy-phenylsulfanyl)-butyl]-piperazine.

LC/MS (m/z) 496 (MH+), Rt=6.80 (method A), purity 78.9%.

5bk. 1-(5-Chloro-2,2-dimethyl-2,3-dihydro-benzofuran-7-yl)-4-[3-(2,6-dichloro-4-fluoro-phenoxy)-propyl]-piperazine.

LC/MS (m/z) 487 (MH+), Rt=6.65 (method A), purity 98.5%.

5bl. 1-(5-Chloro-3,3-dimethyl-2,3-dihydro-benzofuran-7-yl)-4-[3-(2,6-dichloro-4-fluoro-phenoxy)-propyl]-piperazine.

LC/MS (m/z) 488 (MH+), Rt=7.56 (method A), purity 88.2%.

5bm. 1-(4-{4-[4-(2,3-Dihydro-benzo[1,4]dioxin-5-yl)-piperazin-1-yl]-butoxy}-3,5-difluoro-phenyl)-propan-1-one.

LC/MS (m/z) 461 (MH+), Rt=5.50 (method A), purity 72.9%.

5bn. 1-[2-(2-Bromo-4,6-difluoro-phenoxy)-ethyl]-4-(2,3-dihydro-benzo[1,4]dioxin-5-yl)-piperazine.

LC/MS (m/z) 455 (MH+), Rt=5.17 (method A), purity 77.3%.

5bo. 1-[3-(2-Bromo-4,6-difluoro-phenoxy)-propyl]-4-(2,3-dihydro-benzo[1,4]dioxin-5-yl)-piperazine.

LC/MS (m/z) 471 (MH+), Rt=5.34 (method A), purity 98.9%.

5bp. 1-[4-(2,6-Dichloro-4-fluoro-phenoxy)-butyl]-4-(2,3-dihydro-benzo[1,4]dioxin-5-yl)-piperazine.

LC/MS (m/z) 455 (MH+), Rt=5.73 (method A), purity 95.0%.

5bq. 1-(2,3-Dihydro-benzof[1,4]dioxin-5-yl)-4-[3-(2,4,6-tribromo-phenoxy)-propyl]-piperazine.

LC/MS (m/z) 593 (MH+), Rt=6.09 (method A), purity 99.7%.

5br. 1-(4-{3-[4-(2,3-Dihydro-benzo[1,4]dioxin-5-yl)-piperazin-1-yl]-propoxy}-3,5-difluoro-phenyl)-propan-1-one.

LC/MS (m/z) 447 (MH+), Rt=5.20 (method A), purity 99.2%.

5bs. 1-{4-[4-(4-Benzo[b]thiophen-7-yl-piperazin-1-yl) -butoxy]-3,5-difluoro-phenyl}-propan-1-one.

LC/MS (m/z) 459 (MH+), Rt=6.11 (method A), purity 80.0%.

5bt. 1-Benzo[h]thiophen-7-yl-4-[3-(2-bromo-4,6-difluoro-phenoxy)-propyl]-piperazine.

LC/MS (m/z) 467 (MH+), Rt=6.05 (method A), purity 98.7%.

5bu. 1-Benzo[b]thiophen-7-yl-4-[4-(2,6-dichloro-4-fluoro-phenoxy)-butyl/]-piperazine.

LC/MS (m/z) 455 (MH+), Rt=6.36 (method A), purity 96.7%.

5by. 1-Benzo[b]thiophen-7-yl-4-[3-(2,4,6-tribromo-phenoxy)-propyl]-piperazine.

LC/MS (m/z) 591 (MH+), Rt=6.71 (method A), purity 99.6%.

5bw. 1-{4-[3-(4-Benzo[b]thiophen-7-yl-piperazin-1-yl)-propoxy]-3,5-difluoro-phenyl}-propan-1-one.

LC/MS (m/z) 445 (MH+), Rt=5.87 (method A), purity 98.4%.

5bx. 3,5-Dibromo-4-{3-[4-(2,3-dihydro-benzo[1,4]dioxin-5-yl)-piperazin-1-yl]-propoxy}-benzonitrile.

LC/MS (m/z) 538 (MH+), Rt=5.37 (method A), purity 76.8%.

5by. 1-[4-(2,6-Dibromo-4-fluoro-phenoxy)-butyl]-4-(2,3-dihydro-benzo[1,4]dioxin-5-yl)-piperazine.

LC/MS (m/z) 545 (MH+), Rt=5.91 (method A), purity 71.2%.

5bz. 1-[4-(4-Bromo-2,6-difluoro-phenoxy)-butyl]-4-(2,3-dihydro-benzo[1,4]dioxin-5-yl)-piperazine.

LC/MS (m/z) 483 (MH+), Rt=5.76 (method A), purity 91.9%.

5ca. 1-Benzo[b]thiophen-7-yl-4-[3-(2,6-dibromo-4-nitro-phenoxy)-propyl]-piperazine.

LC/MS (m/z) 554 (MH+), Rt=6.24 (method A), purity 87.4%.

5cb. 4-[3-(4-Benzo[b]thiophen-7-yl-piperazin-1-yl)-propoxy]-3,5-dibromo-benzonitrile.

LC/MS (m/z) 538 (MH+), Rt=6.05 (method A), purity 94.1%.

5cc 1-Benzo[b]thiophen-7-yl-4-[4-(4-bromo-2,6-difluoro-phenoxy)-butyl]-piperazine.

LC/MS (m/z) 481 (MH+), Rt=6.34 (method A), purity 94.1%.

5cd. 1-[3-(2-Chloro-phenylsulfanyl)-propyl]-4-(2,3-dihydro-benzo[1,4]dioxin-5-yl)-piperazine.

LC/MS (m/z) 405 (MH+), Rt=5.57 (method A), purity 99.5%.

5ce. 1-Benzo[b]thiophen-7-yl-4-[3-(2-chloro-phenylsulfanyl)-propyl]-piperazine.

LC/MS (m/z) 403 (MH+), Rt=5.99 (method A), purity 100%.

5cf. 1-[3-(2,4-Difluoro-phenoxy)-propyl]-4-(2,3-dihydro-benzo[1,4]dioxin-5-yl)-piperazine.

LC/MS (m/z) 391 (MH+), Rt=7.66 (method A), purity 92.5%.

5cg. 1-[3-(4-Bromo-2,6-difluoro-phenoxy)-propyl]-4-(2,3-dihydro-benzo[1,4]dioxin-5-yl)-piperazine.

LC/MS (m/z) 471 (MH+), Rt=5.53 (method A), purity 97.9%.

5ch. 1-Benzo[b]thiophen-7-yl-4-[2-(2-bromo-4,6-difluoro-phenoxy)-ethyl]-piperazine.

LC/MS (m/z) 455 (MH+), Rt=5.93 (method A), purity 92.0%.

5ci. 1-Benzo[b]thiophen-7-yl-4-[3-(2,4-difluoro-phenoxy)-propyl]-piperazine.

LC/MS (m/z) 389 (MH+), Rt=5.76 (method A), purity 81.7%.

5cj. 1-Benzo[b]thiophen-7-yl-4-[3-(4-bromo-2,6-difluoro-phenoxy)-propyl]-piperazine.

LC/MS (m/z) 469 (MH+), Rt=6.20 (method A), purity 98.5%.

5ck. 8-{4-[3-(2-chloro-4-fluorophenoxy)-propyl]-piperazin-1-yl}-2,3-dihydro-benzo[1,4]dioxine-5-carbonitrile.

LC/MS (m/z) 432 (MH+), Rt=2.29 (method A), purity 75.0%.

5cl. 8-{4-[3-(2,6-Dichloro-phenoxy)-propyl]-piperazin-1-yl}-2,3-dihydro-benzo[1,4]dioxine-5-carbonitrile.

LC/MS (m/z) 464 (MH+), Rt=2.41 (method A), purity 67%.

### Example 6

6a. 8-{4-[3-(4-Fluoro-2-methyl-phenoxy)-propyl]-piperazin-1-yl}-2,3-dihydro-benzo[1,4]dioxine-5-carbonitrile, oxalate. Ethyl 2,3 dihydroxybenzoic acid (103 g) and 1,2-dibromoethane (250 mL) was dissolved in ethanol (1.0 L), to this stirred mixture was a solution of potassium tert-butoxide (316 g) in ethanol (1.5 L) added dropwise over 8 hrs, the reaction was stirred for 16 hrs. 1,2 dibromoethane (100 mL) more was added, and also potassium tert-butoxide (126 g) in ethanol (700 mL) added dropwise and reaction was again stirred for 16 hrs. When the reaction was complete it was filtered and evaporated followed by standard washing procedure from ethylacetate. The crude dioxane (108 g) was obtained sufficiently pure for direct use in the subsequent reaction. 5-Carboxyethyl benzodioxane was dissolved in an ethanol:water mixture (400 mL, 1:1) and sodiumhydroxide (68 mL) was added dropwise at ambient temperature, followed by stirring for 16 hrs. The reaction was evaporated, redissolved in ethylacetate and pH was adjusted to 3, followed by washing using standard procedure to give the free acid (86.5 g).

The acid (229 g) was dissolved in thionyl chloride (2.0 L) and heated at reflux temperature for 3 hrs, and then cooled and evaporated, the remaines was co-evaporated 3 times with toluene. The crude chloride was dissolved in toluene and added dropwise to ammoniumhydroxide solution (1.5 L) at 0° C. Further stirring at room temperature for 30 min gave the full precipitation of the amido-derivative. The precipitated product was filtered and washed (water and ethylacetate) to give the pure amido-derivative (267 g) containing some moisture. This compound was mixted with thionylchloride (1.5 L) and heated at reflux temperature for 7 hrs, cooled, evaporated and co-evaporated with toluene (3 times) followed by standard washing to give the 5-cyano benzodioxane (202 g) as clear pure oil. A part of this cyano derivative (25.5 g) was dissolved in acetic acid (120 mL) and warmed to 60° C., whereafter acetic acid solution (70 mL) of bromine (61 mL) was added dropwise over 15 min. The mixture was heated at 80° C. for 2.5 hrs, cooled and filtered to give the crude crystalline 6,7-dibromo-5-cyano benzodioxane (24.7 g). The obtained dibromo derivative was added portionwise to cooled nitric acid (fuming, 100 mL) at 0° C. over 5 min. After 10 min at room temperature the reaction was poured into icewater (800 mL) and stirred for 30 min. the precipitated product was filtered and dried (25.7 g). The obtained nitro compound was reduced by dissolving it together with potassium hydroxide (11.8 g) in methanol (600 mL). Palladium on charcoal (5%, 21.0 g) was added and the mixture was shaken under a hydrogen pressure (3 bar) for 3 hrs. When all strating material was consumed water was added and mixture was washed using standard procedure into ethylacetate. Evaporation gave the pure 5-amino-8-cyano benzodioxane (12 g) which was dissolved in chlorobenzene (160 mL), and bis-(chloroethyl)amine hydrochloride (12.3 g) was added. The reaction mixture was heated at reflux temperature for 60 hrs, the reaction mixture was cooled and chlorobenzene was decanted of. The crude product was directly dissolved in tetrahydrofuran (500 mL) and water (500 mL) and potassiumcarbonate (92 g) was added, a solution of di tertbutyl carbonate (46.8 g) in tetrahydrofuran (100 mL) was added dropwise to the stirred solution at room temperature. The reaction was stirred for 16 hrs and washed using standard procedure. The obtained crude product was purifyed using silica gel flash chromatography to give the tertbutylcarbamate derivative (25 g). A part of this product (10.9 g) was deprotected by hydrochloride acid-ether treatment to give the pure crystalline amine (8.6 g) as a hydrochloride salt. Treatment of this hydrochloride with ammoniumhydroxide gave the free base, which was washed with ethylacetate using standard procedure. A part of the 1-[8-Cyano-1,4-benzodioxan-5-yl]-piperazine (0.44 g) was dissolved in a mixture of methyl isobutylketone and N,N-dimethylformamide (6+6 mL) followed by addition of potassiumcarbonate (0.48 g), this mixture was stirred for 15 min. 3-(2-chloro-4-fluorophenyl-1-yl)-oxy]propyl bromide (0.53 g) dissolved in methyl isobutylketone (4 mL) was added and the reaction mixture heated to reflux temperature for 1.5 hrs, cooled and evaporated to dryness followed by washing from ethylacetate using standard procedure. The collected pure oil was dissolved in acetone followed by addition of oxalic acid, filtration gave the title compound as pure crystalline material (0.14 g). Mp 118-120° C. ¹H NMR (500 MHz): 2.18 (m, 5H); 2.75-3,00 (m, 6H); 3.35 (m, 4H); 4.00 (t, 2H); 4.35 (dd, 4H); 6,50 (d, 1H); 6.63 (m, 1H); 6.72 (m, 2H); 7,08 (d, 1H); 7,30 (dd, 1H); 7,50 (d, 2H). MS (m/z): 496 (MH+). Anal. Calcd. for C₂₃H₂₆FN₃O₃: C, 58.19; H, 5.80; N, 8.15. Found C, 58.26; H, 5.55; N, 8.50.

6b. 8-{4-[3-(2-Bromo-4-fluoro-phenoxy)-propyl]-piperazin-1-yl}-2,3-dihydro-benzo[1,4]dioxine-5-carbonitrile, oxalate. Mp 152-154° C. ¹H NMR: 2.08 (t, 2H); 3.00 (t, 2H); 3,05 (s, 4H); 3.25 (s, 4H); 4.09 (t, 2H); 4.35 (dd, 4H); 6,60 (d, 1H); 7.18 (m, 3H); 7,55 (d, 1H). MS (m/z): 476 (MH+), 397, 258, 149. Anal. Calcd. for C₂₂H₂₃BrFN₃O₃: C, 50.25; H, 4.54; N, 7.33. Found C, 50.31; H, 4.64; N, 6.85.

(d, 1H). MS (m/z): 476 (MH+), 397, 258, 149. Anal. Calcd. for C₂₂H₂₃BrFN₃O₃: C, 50.25; H, 4.54; N, 7.33. Found C, 50.31; H, 4.64; N, 6.85.

6c. 8-{4-[3-(2-Chloro-phenoxy)-propyl]-piperazin-1-yl}-2,3-dihydro-benzo[1,4]dioxine-5-carbonitrile, oxalate. Mp 96-98° C. ¹H NMR: 2.09 (m, 2H); 2.95-3,05 (m, 6H); 3.28 (m, 4H); 4.12 (s, 2H); 4.38 (dd, 4H); 6,60 (d, 114); 6.95(t, 1H); 7.15-7.23 (m, 2H); 7.30 (t, 1H); 7,43 (d, 1H). MS (m/z): 414 (MH+), 258, 149. Anal. Calcd. for C₂₂H₂₄ClN₃O₃: C, 56.28; H, 5.30; N, 8.21. Found C, 56.22; H, 5.35; N, 8.21.

### Example 7

7aa. 1-(2,3-Dihydro-benzo[1,4]dioxin-5-yl)-4-(2-phenylsulfanyl-ethyl)-piperazine.

To a solution of thiophenol (176 mg, 1.6 mmol) in DMF (1.6 mL) was added a solution of potassium-tert.-butoxide (1.6 mL, 1.6 mmol, 1.0M in tert.-butanol). The mixture was stirred for 5 min. at room temperature. An aliquot of the resulting solution (850 μL) was added to a solution of 2-bromo-1,1-dimethoxyethane (59 mg, 0.35 mmol) in DMF (0.70 mL). The reaction mixture was warmed to 80° C. and stirred for 16 h. After cooling to room temperature, ethyl acetate (6 mL) was added. The organic phase was washed with water (2×4 mL), and dried over sodium sulphate. After evaporation of the volatiles in vacuo, the resulting oil was dissolved in a mixture of dioxane and 3M HCl (4 mL, dioxane:3M HCl 8:1) and heated to 80° C. for 1 h. After cooling to room temperature, ethyl acetate (6 mL) was added. The organic phase was washed with water (2×4 mL), and dried over sodium sulphate. After evaporation of the volatiles in vacuo, the resulting oil was dissolved in 1,2-dichloroethane (1.80 mL). An aliquot of the resulting solution (600 μL) was added to a solution of 1-(2,3-Dihydro-benzo[1,4]dioxin)piperazine (22.4 mmol) in DMF (60 PL), followed by sodium triacetoxyborohydride (30 mg, 0.14 mmol). After shaking the mixture at room temperature for 2 h, a mixture of methanol/water (600 μL, methanol:water 9:1) was added, and the resulting solution was loaded on a pre-conditioned ion exchange column. The column was washed with acetonitrile (2.5 mL) and methanol (2.5 mL), followed by elution of the product with 4 N solution of ammonia in methanol (4.5 mL). After removal of solvents in vacuo, the the title compound was obtained as a colorless oil (5.7 mg, 16.9 μmol, 75%).

LC/MS (m/z) 338 (MH+), Rt=2.07 (method B), purity 89.3%.

The following compounds where prepared analogously:

7ab. 1-(2,3-Dihydro-benzo[1,4]dioxin-5-yl)-4-[2-(2,6-dimethyl-phenoxy)-ethyl]-piperazine.

LC/MS (m/z) 369 (MH+), Rt=2.34 (method B), purity 88.86%.

7ac. 1-(2,3-Dihydro-benzo[1,4]dioxin-5-yl)-4-[4-(2,6-dimethyl-phenylsulfanyl)-butyl]-piperazine.

LC/MS (m/z) 413 (MH+), Rt=2.54 (method B), purity 99.1%

7ad. 1-(2,3-Dihydro-benzo[1,4]dioxin-5-yl)-4-[2-(2,4-dimethyl-phenylsulfanyl)-ethyl]-piperazine.

LC/MS (m/z) 385 (MH+), Rt=2.35 (method B), purity 96.14%

7ae. 1-(2,3-Dihydro-benzo[1,4]dioxin-5-yl)-4-[2-(2-trifluoromethyl-phenoxy)-ethyl]-piperazine.

LC/MS (m/z) 409 (MH+), Rt=2.31 (method B), purity 80.22%

7af. 1-(2,3-Dihydro-benzo[1,4]dioxin-5-yl)-4-[2-(2-trifluoromethyl-phenylsulfanyl)-ethyl]-piperazine.

LC/MS (m/z) 425 (MH+), Rt=2.33 (method B), purity 98.58%

7ag. 1-(2,3-Dihydro-benzo[1,4]dioxin-5-yl)-4-[2-(2-ethyl-phenoxy)-ethyl]-piperazine.

LC/MS (m/z) 369 (MH+), Rt=2.32 (method B), purity 75.61%

7ah. 1-[2-(2,3-Dichloro-phenylsulfanyl)-ethyl]-4-(2,3-dihydro-benzo[1,4]dioxin-5-yl)-piperazine.

LC/MS (m/z) 425 (MH+), Rt=2.38 (method B), purity 97.58%

7ai. 1-[2-(2-Allyl-6-chloro-phenoxy)-ethyl]-4-(2,3-dihydro-benzo[1,4]dioxin-5-yl)-piperazine.

LC/MS (m/z) 415 (MH+), Rt=2.44 (method B), purity 91.16%

7aj. 1-(2,3-Dihydro-benzo[1,4]dioxin-5-yl)-4-[3-(2,4-dimethyl-phenylsulfanyl)-propyl]-piperazine.

LC/MS (m/z) 399 (MH+), Rt=2.43 (method B), purity 95.09%

7ak. 1-(2,3-Dihydro-benzo[1,4]dioxin-5-yl)-4-[3-(2-trifluoromethyl-phenylsulfanyl)-propyl]-piperazine.

LC/MS (m/z) 439 (MH+), Rt=2.4 (method B), purity 93.66%

7al. 1-[3-(2,3-Dichloro-phenylsulfanyl)-propyl]-4-(2,3-dihydro-benzo[1,4]dioxin-5-yl)-piperazine.

LC/MS (m/z) 439 (MH+), Rt=2.47 (method B), purity 94.59%

7am. 1-[3-(3,4-Dichloro-phenylsulfanyl)-propyl]-4-(2,3-dihydro-benzo[1,4]dioxin-5-yl)-piperazine.

LC/MS (m/z) 439 (MH+), Rt=2.52 (method B), purity 94.34%

7an. 1-[4-(3,4-Dichloro-phenylsulfanyl)-butyl]-4-(2,3-dihydro-henzo[1,4]dioxin-5-yl)-piperazine.

LC/MS (m/z) 453 (MH+), Rt=2.62 (method B), purity 72.11%

7ao. 1-[4-(2-Chloro-5-methyl-phenoxy)-butyl]-4-(2,3-dihydro-benzo[1,4]dioxin-5-yl)-piperazine.

LC/MS (m/z) 417 (MH+), Rt=2.27 (method C), purity 84.86%

7ap. 1-[2-(2,4-Dichloro-phenylsulfanyl)-ethyl]-4-(2,3-dihydro-henzo[1,4]dioxin-5-yl)-piperazine.

LC/MS (m/z) 425 (MH+), Rt=2.17 (method C), purity 93.15%

7aq . 1-(2,3-Dihydro-benzo[1,4]dioxin-5-yl)-4-(3-m-tolylsulfanyl-propyl)-piperazine.

LC/MS (m/z) 385 (MH+), Rt=2.05 (method C), purity 75.1%

7ar. 1-[4-(2,4-Dichloro-phenylsulfanyl)-butyl]-4-(2,3-dihydro-benzo[1,4]dioxin-5-yl)-piperazine.

LC/MS (m/z) 453 (MH+), Rt=2.37 (method C), purity 73.44%

7as. 1-(2,3-Dihydro-benzo[1,4]dioxin-5-yl)-4-[2-(2-ethyl-phenylsulfanyl)-ethyl]-piperazine.

LC/MS (m/z) 385 (MH+), Rt=2.09 (method C), purity 96.15%

7at. 1-[2-(2,5-Dichloro-phenylsulfanyl)-ethyl]-4-(2,3-dihydro-henzo[1,4]dioxin-5-yl)-piperazine.

LC/MS (m/z) 425 (MH+), Rt=2.11 (method C), purity 96.58%

7au. 1-[2-(3-Chloro-phenylsulfanyl)-ethyl]-4-(2,3-dihydro-benzo[1,4]dioxin-5-yl)-piperazine.

LC/MS (m/z) 391 (MH+), Rt=1.99 (method C), purity 95.76%

7av. 1-[2-(2-Chloro-phenylsulfanyl)-ethyl]-4-(2,3-dihydro-benzo[1,4]dioxin-5-yl)-piperazine.

LC/MS (m/z) 391 (MH+), Rt=1.92 (method C), purity 97.93%

7aw. 1-(2,3-Dihydro-benzo[1,4]dioxin-5-yl)-4-[2-(2-fluoro-phenylsulfanyl)-ethyl]-piperazine.

LC/MS (m/z) 375 (MH+), Rt=1.82 (method C), purity 94.32%

7ax. 1-(2,3-Dihydro-benzo[1,4]dioxin-5-yl)-4-[3-(2-ethyl-phenylsulfanyl)-propyl]-piperazine.

LC/MS (m/z) 399 (MH+), Rt=2.17 (method C), purity 83.64%

7ay. 1-[3-(2,5-Dichloro-phenylsulfanyl)-propyl]-4-(2,3-dihydro-benzo[1,4]dioxin-5-yl)-piperazine.

LC/MS (m/z) 439 (MH+), Rt=2.19 (method C), purity 89.61%

7az. 1-[3-(3-Chloro-phenylsulfanyl)-propyl]-4-(2,3-dihydro-benzo[1,4]dioxin-5-yl)-piperazine.

LC/MS (m/z) 405 (MH+), Rt=2.09 (method C), purity 87.22%

7ba. 1-(2,3-Dihydro-benzo[1,4]dioxin-5-yl)-4-[3-(2-fluoro-phenylsulfanyl)-propyl]-piperazine.

LC/MS (m/z) 389 (MH+), Rt=1.91 (method C), purity 85.93%

7bb. 3-Chloro-4-{4-[4-(2,3-dihydro-benzo[1,4]dioxin-5-yl)-piperazin-1-yl]-butoxy}-benzonitrile.

LC/MS (m/z) 428 (MH+), Rt=1.95 (method C), purity 76.61%

7bc. 1-(2,3-Dihydro-benzo[1,4]dioxin-5-yl)-4-(4-o-tolylsulfanyl-butyl)-piperazine.

LC/MS (m/z) 399 (MH+), Rt=2.13 (method C), purity 72.93%

7bd. 1-[4-(2,5-Dichloro-phenylsulfanyl)-butyl]-4-(2,3-dihydro-benzo[1,4]dioxin-5-yl)-piperazine.

LC/MS (m/z) 453 (MH+), Rt=2.31 (method C), purity 77.14%

7be. 1-[4-(2-Chloro-phenylsulfanyl)-butyl]-4-(2,3-dihydro-benzo[1,4]dioxin-5-yl)-piperazine.

LC/MS (m/z) 419 (MH+), Rt=2.14 (method C), purity 75.5%

7bf. 1-(2,3-Dihydro-benzo[1,4]dioxin-5-yl)-4-[4-(2-fluoro-phenylsulfanyl)-butyl]-piperazine.

LC/MS (m/z) 403 (MH+), Rt=2.03 (method C), purity 74.97%

7bg. 1-(2,3-Dihydro-benzo[1,4]dioxin-5-yl)-4-[2-(3,4-dimethoxy-phenylsulfanyl)-ethyl]-piperazine.

LC/MS (m/z) 417 (MH+), Rt=1.7 (method D), purity 89.79%

7bh. 3-{4-[4-(2,3-Dihydro-benzo[1,4]dioxin-5-yl)-piperazin-1-yl]-butoxy}-benzonitrile.

LC/MS (m/z) 394 (MH+), Rt=1.85 (method D), purity 75.52%

7bi. 1-[4-(2-Chloro-4-fluoro-phenylsulfanyl)-butyl]-4-(2,3-dihydro benzo[1,4]dioxin-5-yl)-piperazine.

LC/MS (m/z) 437 (MH+), Rt=2.23 (method D), purity 86.05%

7bj. 1-(2,3-Dihydro-benzo[1,4]dioxin-5-yl)-4-[3-(4-trifluoromethoxy-phenylsulfanyl)-propyl]-piperazine.

LC/MS (m/z) 455 (MH+), Rt=2.29 (method D), purity 86.83%

7bk. 1-(2,3-Dihydro-benzo[1,4]dioxin-5-yl)-4-[3-(2,5-dimethoxy-phenylsulfanyl)-propyl]-piperazine.

LC/MS (m/z) 431 (MH+), Rt=1.9 (method D), purity 74.89%

7bl. 1-(2,3-Dihydro-benzo[1,4]dioxin-5-yl)-4-[3-(3-bromo-phenylsulfanyl)-propyl]-piperazine.

LC/MS (m/z) 449 (MH+), Rt=2.13 (method D), purity 88.56%

7bm. 1-(2,3-Dihydro-benzo[1,4]dioxin-5-yl)-4-[4-(2-methoxy-phenylsulfanyl)-butyl]-piperazine.

LC/MS (m/z) 415 (MH+), Rt=1.94 (method C), purity 94,04

7bn. 1-(2,3-Dihydro-benzo[1,4]dioxin-5-yl)-4-[4-(2-isopropyl-phenylsulfanyl)-butyl]-piperazine.

LC/MS (m/z) 427 (MH+), Rt=2.39 (method C), purity 73,56

7bo. 1-(2,3-Dihydro-benzo[1,4]dioxin-5-yl)-4-(2-o-tolylsulfanyl-ethyl)-piperazine.

LC/MS (m/z) 371 (MH+), Rt=1.92 (method C), purity 93,93

7bp. 1-[4-(2-Allyl-phenoxy)-butyl]-4-(2,3-dihydro-benzo[1,4]dioxin-5-yl)-piperazine.

LC/MS (m/z) 409 (MH+), Rt=2.26 (method C), purity 91,57

Pharmacological Testing

The affinity of the compounds of the invention to 5-HT_{1A} receptors was determined by measuring the inhibition of binding of a radioactive ligand at 5-HT_{1A} receptors as described in the following test:

Inhibition of ³H-5-CT Binding to Human 5-HT_{1A} Receptors

By this method the inhibition by drugs of the binding of the 5-HT_{1A} agonist ³H-5-carboxamido tryptamine (3H-5-CT) to cloned human 5-HT_{1A} receptors stably expressed in transfected HeLa cells (HA7) (Fargin, A. et al, J. Biol. Chem., 1989, 264, 14848) is determined in vitro. The assay was performed as a modification of the method described by Harrington, M. A. et at, J. Pharmacol. Exp. Ther., 1994, 268, 1098. Human 5-HT_{1A} receptors (40 μg of cell homogenate) were incubated for 15 minutes at 37° C. in 50 mM Tris buffer at pH 7.7 in the presence of ³H-5-CT. Non-specific binding was determined by including 10 μM of metergoline. The reaction was terminated by rapid filtration through Unifilter GF/B filters on a Tomtec Cell Harvester. Filters were counted in a Packard Top Counter. The results obtained are presented in table 1.

The compounds of the invention have also been tested for their affinity to dopamine D₄ receptors in the following test.

Inhibition of the Binding of ³H-YM-09151-2 to Human Dopamine D₄ Receptors

By this method the inhibition by drugs of the binding of [³H]YM-09151-2 (0.06 nM) to membranes of human cloned dopamine D_{4.2} receptors expressed in CHO-cells is determined in vitro. Method modified from NEN Life Science Products, Inc., technical data certificate PC2533-10/96.

The results are given in the following Table 1 as IC₅₀-values.

TABLE 1
Binding Data

| | Inhibition of | Inhibition of | | Inhibition of | Inhibition of |
|---|---|---|---|---|---|
| | ³H-5-CT Binding | ³H-YM-09151-2 binding | | ³H-5-CT Binding | ³H-YM-09151-2 binding |
| Compound | IC₅₀ (nM) or % | IC₅₀ (nM) or % | Compound | IC₅₀ (nM) or % | IC₅₀ (nM) or % |
| No. | inhibition at 100 nM | inhibition at 50 nM | No. | inhibition at 100 nM | inhibition at 50 nM |
| 1a | 10. | 1.1 | 5ci | 3.5 | 2.0 |
| 1b | 1.1 | 5.9 | 5cj | 93% | 59. |
| 1c | 2.0 | 13 | 5ck | 180 | 77% |
| 1d | 5.3 | 3.0 | 5cl | 83% | 42% |
| 1e | 4.5 | 1.3 | 6a | 120 | 6.9 |
| 2a | 4.4 | 4.0 | 6b | 230 | 10 |
| 2b | 15. | 12. | 6c | 68 | 13 |
| 2c | 4.0 | 1.2 | 7aa | 78% | 84% |
| 2d | 15. | 1.7 | 7ab | 86% | 91% |
| 2e | 3.0 | 5.4 | 7ac | 96% | 96% |
| 3a | 76. | 5.4 | 7ad | 21 | 91% |
| 3b | 97. | 4.3 | 7ae | 82% | 75% |
| 3c | 11. | 35 | 7af | 99% | 91% |
| 3d | 31. | 11 | 7ag | 79% | 88% |
| 4a | 2.8 | 1.3 | 7ah | 1.6 | 87% |
| 5aa | 4.9 | 0.53 | 7ai | 90% | 82% |
| 5ab | 1.9 | 2.1 | 7aj | 99% | 84% |
| 5ac | 2.4 | 1.4 | 7ak | 93% | 98% |
| 5ad | 15. | 6.6 | 7al | 98% | 97% |
| 5ag | 7.4 | 3.1 | 7am | 100% | 99% |
| 5ai | 17. | 2.9 | 7an | 100% | 91% |
| 5aj | 3.4 | 13. | 7ao | 97% | 89% |
| 5ao | 3.8 | 68. | 7ap | 91% | 78% |
| 5ap | 6.2 | 6.3 | 7aq | 101% | 82% |
| 5at | 2.2 | 3.4 | 7ar | 3.8 | 99% |
| 5au | 6.4 | 7.6 | 7as | 80% | 93% |
| 5av | 5.1 | 1.4 | 7at | 88% | 92% |
| 5aw | 1.7 | 1.9 | 7au | 84% | 87% |
| 5ax | 33% | 23. | 7av | 90% | 75% |
| 5ay | 14% | 12. | 7aw | 74% | 92% |
| 5az | 18% | 9.9 | 7ax | 9.2 | 100% |
| 5bc | 53. | 1.9 | 7ay | 2.0 | 102% |
| 5bd | 52. | 6.5 | 7az | 100% | 92% |
| 5be | 8.0 | 1.2 | 7ba | 97% | 84% |
| 5bf | 35. | 3.1 | 7bb | 101% | 94% |
| 5bh | 1.7 | 76. | 7bc | 82% | 96% |
| 5bi | 3.5 | 87. | 7bd | 84% | 102% |
| 5bk | 22. | 11. | 7be | 102% | 102% |
| 5bl | 88. | 26. | 7bf | 101% | 91% |
| 5bo | 1.7 | 2.4 | 7bg | 81% | 64% |
| 5bp | 4.8 | 6.2 | 7bh | 95% | 84% |
| 5bq | 1.2 | 2.4 | 7bi | 96% | 101% |
| 5bt | 8.6 | 6.3 | 7bj | 106% | 92% |
| 5bx | 2.2 | 19. | 7bk | 91% | 84% |
| 5by | 2.5 | 4.3 | 7bl | 95% | 102% |
| 5bz | 5.0 | 10. | 7bm | 95% | 83% |
| 5cc | 9.7 | 34. | 7bn | 93% | 93% |
| 5cd | 8.8 | 2.3 | 7bo | 91% | 102% |
| 5ce | 4.8 | 16. | 7bp | 92% | 99% |
| 5cf | 4.9 | 1.4 | — | — | — |
| 5cg | 1.7 | 12. | | | |

The compounds of the invention have also been tested for their effect on re-uptake of serotonin in the following test:

Inhibition of ³H-5-HT Uptake Into Rat Brain Synaptosomes

Using this method, the ability of drugs to inhibit the accumulation of ³H-5-HT into whole rat brain synaptosomes is determined in vitro. The assay was performed as described by Hyttel, J., Psychopharmacology 1978, 60, 13.

Furthermore, the 5-HT_{1A} antagonistic activity of some of the compounds of the invention has been estimated in vitro at cloned 5-HT_{1A} receptors stably expressed in transfected HeLa cells (HA7). In this test, 5-HT_{1A} antagonistic activity is estimated by measuring the ability of the compounds to antagonize the 5-HT induced inhibition of forskolin induced cAMP accumulation. The assay was performed as a modification of the method described by Pauwels, P. J. et al, Biochem. Pharmacol. 1993, 45, 375.

The compounds of the invention have also been tested for their affinity to dopamine D₃ receptors in the following test.

Inhibition of the Binding of [³H]-Spiperone to Human D₃ Receptors

By this method the inhibition by drugs of the binding [³H]Spiperone (0.3 nM) to membranes of human cloned dopamine D₃ receptors expressed in CHO-cells is determined in vitro. Method modified from R. G. MacKenzie et al., Eur. J. Pharm. Mol. Pharm. Sec., 1994, 266, 79-85.

As seen from the above, the compounds of the invention show affinity for the 5-HT_{1A} receptors and for dopamine D₄ receptors. Furthermore, many of the compounds of the present invention possess valuable activity as serotonin re-uptake inhibitors and/or have effect at dopamine D₃ receptors. Accordingly, the compounds are considered useful for the treatment of psychiatric and neurological disorders as mentioned previously.

Pharmaceutical Formulation

The pharmaceutical formulations of the invention may be prepared by conventional methods in the art. For example: Tablets may be prepared by mixing the active ingredient with ordinary adjuvants and/or diluents and subsequently compressing the mixture in a conventional tabletting machine. Examples of adjuvants or diluents comprise: corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients. Solutions for injections may be prepared by dissolving the active ingredient and possible additives in a part of the solvent for injection, preferably sterile water, adjusting the solution to desired volume, sterilisation of the solution and filling in suitable ampules or vials. Any suitable additive conventionally used in the art may be added, such as tonicity agents, preservatives, antioxidants, etc.

The pharmaceutical compositions of this invention or those which are manufactured in accordance with this invention may be administered by any suitable route, for example orally in the form of tablets, capsules, powders, syrups, etc., or parenterally in the form of solutions for injection. For preparing such compositions, methods well known in the art may be used, and any pharmaceutically acceptable carriers, diluents, excipients, or other additives normally used in the art may be used.

Conveniently, the compounds of the invention are administered in unit dosage form containing said compounds in an amount of about 0.01 to 1000 mg. The total daily dose is usually in the range o f about 0.05-500 mg, and most preferably about 0.1 to 50 mg of the active compound of the invention.

1. A heteroaryl derivative having the general Formula I: 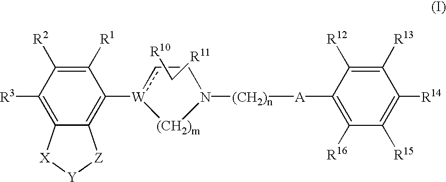
any of its enantiomers or any mixture thereof, or an acid addition salt thereof, wherein
X is —O—, —S—, or —CR⁴R⁵—; and
Y is —CR⁶R⁷—, —CR⁶R⁷—CR⁸R⁹—, or —CR⁶CR⁷—; or
X and Y together form a group —CR⁴═CR⁵—, or —CR⁴═CR⁵—CR⁶R⁷—;
Z is —O—, or —S—;
W is N, C, or CH;
n is 2, 3, 4, 5, 6, 7, 8, 9 or 10;
m is 2 or 3:
A is O or S
wherein the dotted lines mean an optional bond;
R¹, R² and R³ are each independently selected from hydrogen, halogen, nitro, cyano, trifluoromethyl, trifluoromethoxy, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylthio, hydroxy, formyl, acyl, amino, C₁₋₆-alkylamino, di(C₁₋₆-alkyl)amino, acylamino, C₁₋₆-alkoxycarbonylamino, aminocarbonylamino, C₁₋₆-alkylaminocarbonylamino and di(C₁₋₆-alkyl)aminocarbonylamino;
R⁴, R⁵, R⁶, R⁷, R⁸ and R⁹ are each independently selected from hydrogen, halogen, trifluoromethyl, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylthio, amino, C₁₋₆-alkylamino, di(C₁₋₆-alkyl)amino, phenylamino or phenyl-C₁₋₆-alkylamino wherein the phenyl group may be substituted, acylamino, hydroxy, —SH, cyano, nitro, —COOR¹⁸, —SO₂—R¹⁹ or
C₁₋₆-alkyl substituted with a substituent selected from halogen, C₁₋₆-alkoxy, C₁₋₆-alkylthio, amino, C₁₋₆-alkylamino, di(C₁₋₆-alkyl)amino, acylamino, hydroxy, —SH, cyano, nitro, —COOR¹⁸ or —SO₂—R¹⁹;
R¹⁸ is hydrogen, C₁₋₆-alkyl, C₂₋₆-alkenyl, C₂₋₆-alkynyl, phenyl or phenyl-C₁₋₆-alkyl wherein the phenyl groups may be substituted, amino, C₁₋₆-alkylamino or di(C₁₋₆-alkyl)amino, and
R¹⁹ is hydrogen, C₁₋₆-alkyl, amino, C₁₋₆-alkylamino, di(C₁₋₆-alkyl)amino, phenyl or phenyl-C₁₋₆-alkyl wherein the phenyl groups may be substituted;
R¹⁰ and R¹¹ are each independently selected from hydrogen and C₁₋₆-alkyl; and
R¹², R¹³, R¹⁴, R¹⁵ and R¹⁶ are each independently selected from hydrogen, halogen, nitro, cyano, trifluoromethyl, trifluoromethoxy, C₁₋₆-alkyl, C₂₋₆-alkenyl, C₂₋₆-alkynyl, C₃₋₈-cycloalkyl, C₃₋₈-cycloalkyl-C₁₋₆-alkyl, C₁₋₆-alkoxy, C₁₋₆-alkylthio, C₁₋₆-alkylsulphonyl, hydroxy, formyl, acyl, amino, acylamino, C₁₋₆-alkoxycarbonylamino, aminocarbonylamino, C₁₋₆-alkylaminocarbonylamino, di(C₁₋₆-alkyl)aminocarbonylamino and NR²⁰R²¹ wherein R²⁰ and R²¹ independently represent hydrogen, C₁₋₆-alkyl, C₃₋₈-cycloalkyl, or phenyl; or R²⁰ and R²¹ together with the nitrogen to which they are attached form a 5- or 6-membered carbocyclic ring optionally containing one further heteroatom;
provided that when X-Y-Z together with the phenyl ring forms a benzofuran or a benzodioxan ring; and A is O, then at least one of R¹², R¹³, R¹⁴, R¹⁵ and R¹⁶ is not hydrogen.

2. A compound of claim 1, wherein
X is —O—; and
Y is —CR⁶R⁷—CR⁸R⁹—; and
Z is —O—.

3. A compound of claim 1, wherein
X is —CR⁴R⁵—; and
Y is —CR⁶R⁷; and
Z is —O—.

4. A compound of claim 1, wherein
X and Y together form a group —CR⁴═CR⁵—; and
Z is —S—.

5. A compound of claim 1, wherein W is N.

6. A compound of claim 1, wherein R¹, R² and R³ are hydrogen;

7. A compound of claim 1, wherein A is O.

8. A compound of claim 1, wherein A is S.

9. A compound of claim 1, wherein n is 2, 3 or 4;

10. A compound of claim 1, wherein R¹², R¹³, R¹⁴, R¹⁵ and R¹⁶ are independently selected from the group consisting of hydrogen, halogen, C₁₋₆-alkyl, C₂₋₆-alkenyl, C₁₋₆-alkoxy, cyano, C₁₋₆-alkylsulphonyl, acyl, nitro, trifluoromethyl, and trifluoromethxoy.

11. A compound of claim 1, wherein at least one of R¹², R¹³, R¹⁴, R¹⁵ and R¹⁶ is halogen.

12. A compound of claim 11, wherein at least one of R¹², R¹³, R¹⁴, R¹⁵ and R¹⁶ is halogen, and the other substituents are selected from the group consisting of hydrogen, halogen, C₁₋₆-alkoxy, C₁₋₆-alkyl, C₂₋₆-alkenyl, C₁₋₆-alkylsulfonyl, acyl, nitro, cyano and trifluoromethyl;.

13. The compound according to claim 1 which is selected from the group consisting of
1-[3-(2-Chloro-phenoxy)-propyl]-4-(2,3-dihydro-benzo[1,4]dioxin-5-yl)-piperazine;
1-[3-(2,6-Dichloro-phenoxy)-propyl]-4-(2,3-dihydro-benzo[1,4]dioxin-5-yl)-piperazine;
1-(2,3-Dihydro-benzo[1,4]dioxin-5-yl)-4-[3-(2,4,6-trifluoro-phenoxy)-propyl]-piperazine;
1-(2,3-Dihydro-benzo[1,4]dioxin-5-yl)-4-[3-(4-fluoro-2-methoxy-phenoxy)-propyl]-piperazine;
1-(2,3-Dihydro-benzo[1,4]dioxin-5-yl)-4-[3-(4-fluoro-2-methyl-phenoxy)-propyl]-piperazine;
1-[3-(4-Chloro-phenoxy)-propyl]-4-(2,3-dihydro-benzo[1,4]dioxin-5-yl)-piperazine;
1-(2,3-Dihydro-benzo[1,4]dioxin-5-yl)-4-[3-(4-trifluoromethyl-phenoxy)-propyl]-piperazine;
1-(2,3-Dihydro-benzo[1,4]dioxin-5-yl)-4-[3-(2-fluoro-phenoxy)-propyl]-piperazine;
2-{3-[4-(2,3-Dihydro-benzo[1,4]dioxin-5-yl)-piperazin-1-yl]-propoxy}-benzonitrile;
1-Benzo[b]thiophen-7-yl-4-[3-(2-chloro-4-fluoro-phenylsulfanyl)-propyl]-piperazine;
1-Benzo[b]thiophen-7-yl-4-[4-(2-chloro-4-fluoro-phenoxy)-butyl]-piperazine;
1-[2-(3,4-Dichloro-phenylsulfanyl)-ethyl]-4-(2,3-dihydro-benzo[1,4]dioxin-5-yl)-piperazine;
1-(2,3-Dihydro-benzo[1,4]dioxin-5-yl)-4-[2-(4-fluoro-phenylsulfanyl)-ethyl]-piperazine;
1-[2-(Bromo-trifluoromethyl-phenylsulfanyl)-ethyl]-4-(2,3-dihydro-benzo[1,4]dioxin-5-yl)-piperazine;
1-[2-(2,6-Dichloro-phenylsulfanyl)-ethyl]-4-(2,3-dihydro-benzo[1,4]dioxin-5-yl)-piperazine;
1-(2,3-Dihydro-benzo[1,4]dioxin-5-yl)-4-(3-phenylsulfanyl-propyl)-piperazine;
1-[3-(2-Bromo-4-fluoro-phenoxy)-propyl]-4-(2,3-dihydro-benzo[1,4]dioxin-5-yl)-piperazine;
1-[4-(2,6-Dichloro-phenylsulfanyl)-butyl]-4-(2,3-dihydro-benzo[1,4]dioxin-5-yl)-piperazine;
1-[3-(2-Chloro-4-fluoro-phenylsulfanyl)-propyl]-4-(2,3-dihydro-benzo[1,4]dioxin-5-yl)-piperazine;
1-Benzo[b]thiophen-7-yl-4-[3-(2-chloro-4-fluoro-phenylsulfanyl)-propyl]-piperazine;
1-Benzo[b]thiophen-7-yl-4-[3-(2,6-dichloro-phenylsulfanyl)-propyl]-piperazine;
1-Benzo[b]thiophen-7-yl-4-[4-(2,6-dichloro-phenylsulfanyl)-butyl]-piperazine;
1-[4-(3-Chloro-2-methoxy-phenylsulfanyl)-butyl]-4-(2,3-dihydro-benzo[1,4]dioxin-5-yl)-piperazine;
1-Benzo[b]thiophen-7-yl-4-[4-(3-chloro-2-methoxy-phenylsulfanyl)-butyl]-piperazine;
1-Benzo[b]thiophen-7-yl-4-[3-(2-chloro-4-fluoro-phenylsulfanyl)-propyl]-piperazine;
1-[3-(2,6-Dibromo-4-fluoro-phenoxy)-propyl]-4-(2,3-dihydro-benzo[1,4]dioxin-5-yl)-piperazine;
1-Benzo[b]thiophen-7-yl-4-[3-(2,6-dibromo-4-fluoro-phenoxy)-propyl]-piperazine;
4-{3-[4-(2,3-Dihydro-benzo[1,4]dioxin-5-yl)-piperazin-1-yl]-propoxy}-3,5-diiodo-benzonitrile;
3,5-Di-tert-butyl-4-{3-[4-(2,3-dihydro-benzo[1,4]dioxin-5-yl)-piperazin-1-yl]-propoxy}-benzonitrile;
1-[3-(2,6-Dichloro-4-methanesulfonyl-phenoxy)-propyl]-4-(2,3-dihydro-benzo[1,4]dioxin-5-yl)-piperazine;
1-Benzo[b]thiophen-7-yl-4-[3-(2,6-dichloro-4-methanesulfonyl-phenoxy)-propyl]-piperazine;
1-[3-(Bromo-trifluoromethyl-phenylsulfanyl)-propyl]-4-(2,3-dihydro-benzo[1,4]dioxin-5-yl)-piperazine;
1-Benzo[b]thiophen-7-yl-4-[3-(bromo-trifluoromethyl-phenylsulfanyl)-propyl]-piperazine;
1-Benzo[b]thiophen-7-yl-4-[4-(2-chloro-6-methyl-phenylsulfanyl)-butyl]-piperazine;
1-Benzo[b]thiophen-7-yl-4-[4-(2-chloro-4-fluoro-phenylsulfanyl)-butyl]-piperazine;
1-[3-(2,6-Dichloro-4-fluoro-phenoxy)-propyl]-4-(2,3-dihydro-benzo[1,4]dioxin-5-yl)-piperazine;
1-Benzo[b]thiophen-7-yl-4-[3-(2,6-dichloro-4-fluoro-phenoxy)-propyl]-piperazine;
1-[4-(2-Chloro-6-methyl-phenylsulfanyl)-butyl]-4-(2,3-dihydro-benzo[1,4]dioxin-5-yl)-piperazine;
1-[3-(2,6-Dichloro-phenylsulfanyl)-propyl]-4-(2,3-dihydro-benzo[1,4]dioxin-5-yl)-piperazine;
1-(5-Chloro-2,2-dimethyl-2,3-dihydro-benzofuran-7-yl)-4-[4-(2-chloro-6-methyl-phenylsulfanyl)-butyl]-piperazine;
1-(5-Chloro-3,3-dimethyl-2,3-dihydro-benzofuran-7-yl)-4-[4-(2-chloro-6-methyl-phenylsulfanyl)-butyl]-piperazine;
1-(5-Chloro-2,2-dimethyl-2,3-dihydro-benzofuran-7-yl)-4-[3-(2,6-dichloro-phenylsulfanyl)-propyl]-piperazine;
1-(5-Chloro-3,3-dimethyl-2,3-dihydro-benzofuran-7-yl)-4-[3-(2,6-dichloro-phenylsulfanyl)-propyl]-piperazine;
1-(5-Chloro-3,3-dimethyl-2,3-dihydro-benzofuran-7-yl)-4-[3-(2-chloro-4-fluoro-phenylsulfanyl)-propyl]-piperazine;
1-Benzo[b]thiophen-7-yl-4-[4-(2-chloro-4-fluoro-phenoxy)-butyl]-piperazine;
1-(5-Chloro-2,2-dimethyl-2,3-dihydro-benzofuran-7-yl)-4-[4-(2-chloro-4-fluoro-phenoxy)-butyl]-piperazine;
1-[4-(2-Bromo-4-fluoro-phenoxy)-butyl]-4-(2,3-dihydro-benzo[1,4]dioxin-5-yl)-piperazine;
1-Benzo[b]thiophen-7-yl-4-[4-(2-bromo-4-fluoro-phenoxy)-butyl]-piperazine;
1-[4-(2-Bromo-4-fluoro-phenoxy)-butyl]-4-(5-chloro-2,2-dimethyl-2,3-dihydro-benzofuran-7-yl)-piperazine;
1-(5-Chloro-2,2-dimethyl-2,3-dihydro-benzofuran-7-yl)-4-[3-(2,6-dichloro-4-methanesulfonyl-phenoxy)-propyl]-piperazine;
1-(5-Chloro-3,3-dimethyl-2,3-dihydro-benzofuran-7-yl)-4-[3-(2,6-dichloro-4-methanesulfonyl-phenoxy)-propyl]-piperazine;
1-(5-Chloro-3,3-dimethyl-2,3-dihydro-benzofuran-7-yl)-4-[4-(3-chloro-2-methoxy-phenylsulfanyl)-butyl]-piperazine;
1-(-Chloro-2,2-dimethyl-2,3-dihydro-benzofuran-7-yl)-4-[3-(2,6-dichloro-4-fluoro-phenoxy)-propyl]-piperazine;
1-(5-Chloro-3,3-dimethyl-2,3-dihydro-benzofuran-7-yl)-4-[3-(2,6-dichloro-4-fluoro-phenoxy)-propyl]-piperazine;
1-(4-{4-[4-(2,3-Dihydro-benzo[1,4]dioxin-5-yl)-piperazin-1-yl]-butoxy}-3,5-difluoro-phenyl)-propan-1-one;
1-[2-(2-Bromo-4,6-difluoro-phenoxy)-ethyl]-4-(2,3-dihydro-benzo[1,4]dioxin-5-yl)-piperazine;
1-[3-(2-Bromo-4,6-difluoro-phenoxy)-propyl]-4-(2,3-dihydro-benzo[1,4]dioxin-5-yl)-piperazine;
1-[4-(2,6-Dichloro-4-fluoro-phenoxy)-butyl]-4-(2,3-dihydro-benzo[1,4]dioxin-5-yl)-piperazine;
1-(2,3-Dihydro-benzo[1,4]dioxin-5-yl)-4-[3-(2,4,6-tribromo-phenoxy)-propyl]-piperazine;
1-(4-{3-[4-(2,3-Dihydro-benzo[1,4]dioxin-5-yl)-piperazin-1-yl]-propoxy}-3,5-difluoro-phenyl)-propan-1-one;
1-{4-[4-(4-Benzo[b]thiophen-7-yl-piperazin-1-yl)-butoxy]-3,5-difluoro-phenyl}-propan-1-one;
1-Benzo[b]thiophen-7-yl-4-[3-(2-bromo-4,6-difluoro-phenoxy)-propyl]-piperazine;
1-Benzo[b]thiophen-7-yl-4-[4-(2,6-dichloro-4-fluoro-phenoxy)-butyl]-piperazine;
1-Benzo[b]thiophen-7-yl-4-[3-(2,4,6-tribromo-phenoxy)-propyl]-piperazine;
1-{4-[3-(4-Benzo[b]thiophen-7-yl-piperazin-1-yl)-propoxy]-3,5-difluoro-phenyl}-propan-1-one;
3,5-Dibromo-4-{3-[4-(2,3-dihydro-benzo[1,4]dioxin-5-yl)-piperazin-1-yl]-propoxy}-benzonitrile;
1-[4-(2,6-Dibromo-4-fluoro-phenoxy)-butyl]-4-(2,3-dihydro-benzo[1,4]dioxin-5-yl)-piperazine;
1-[4-(4-Bromo-2,6-difluoro-phenoxy)-butyl]-4-(2,3-dihydro-benzo[1,4]dioxin-5-yl)-piperazine;
1-Benzo[b]thiophen-7-yl-4-[3-(2,6-dibromo-4-nitro-phenoxy)-propyl]-piperazine;
4-[3-(4-Benzo[b]thiophen-7-yl-piperazin-1-yl)-propoxy]-3,5-dibromo-benzonitrile;
1-Benzo[b]thiophen-7-yl-4-[4-(4-bromo-2,6-difluoro-phenoxy)-butyl]-piperazine;
1-[3-(2-Chloro-phenylsulfanyl)-propyl]-4-(2,3-dihydro-benzo[1,4]dioxin-5-yl)-piperazine;
1-Benzo[b]thiophen-7-yl-4-[3-(2-chloro-phenylsulfanyl)-propyl]-piperazine;
1-[3-(2,4-Difluoro-phenoxy)-propyl]-4-(2,3-dihydro-benzo[1,4]dioxin-5-yl)-piperazine;
1-[3-(4-Bromo-2,6-difluoro-phenoxy)-propyl]-4-(2,3-dihydro-benzo[1,4]dioxin-5-yl)-piperazine;
1-Benzo[b]thiophen-7-yl-4-[2-(2-bromo-4,6-difluoro-phenoxy)-ethyl]-piperazine;
1-Benzo[b]thiophen-7-yl-4-[3-(2,4-difluoro-phenoxy)-propyl]-piperazine;
1-Benzo[b]thiophen-7-yl-4-[3-(4-bromo-2,6-difluoro-phenoxy)-propyl]-piperazine;
8-{4-[3-(2-chloro-4-fluorophenoxy)-propyl]-piperazin-1-yl}-2,3-dihydro-benzo[1,4]dioxine-5-carbonitrile;
8-{4-[3-(2,6-Dichloro-phenoxy)-propyl]-piperazin-1-yl}-2,3-dihydro-benzo[1,4]dioxine-5-carbonitrile;
8-{4-[3-(4-Fluoro-2-methyl-phenoxy)-propyl]-piperazin-1-yl}-2,3-dihydro-benzo[1,4]dioxine-5-carbonitrile;
8-{4-[3-(2-Bromo-4-fluoro-phenoxy)-propyl]-piperazin-1-yl}-2,3-dihydro-benzo[1,4]dioxine-5-carbonitrile;
8-{4-[3-(2-Chloro-phenoxy)-propyl]-piperazin-1-yl}-2,3-dihydro-benzo[1,4]dioxine-5-carbonitrile;
1-(2,3-Dihydro-benzo[1,4]dioxin-5-yl)-4-(2-phenylsulfanyl-ethyl)-piperazine;
1-(2,3-Dihydro-benzo[1,4]dioxin-5-yl)-4-[2-(2,6-dimethyl-phenoxy)-ethyl]-piperazine;
1-(2,3-Dihydro-benzo[1,4]dioxin-5-yl)-4-[4-(2,6-dimethyl-phenylsulfanyl)-butyl]-piperazine;
1-(2,3-Dihydro-benzo[1,4]dioxin-5-yl)-4-[2-(2,4-dimethyl-phenylsulfanyl)-ethyl]-piperazine;
1-(2,3-Dihydro-benzo[1,4]dioxin-5-yl)-4-[2-(2-trifluoromethyl-phenoxy)-ethyl]-piperazine;
1-(2,3-Dihydro-benzo[1,4]dioxin-5-yl)-4-[2-(2-trifluoromethyl-phenylsulfanyl)-ethyl]-piperazine;
1-(2,3-Dihydro-benzo[1,4]dioxin-5-yl)-4-[2-(2-ethyl-phenoxy)-ethyl]-piperazine;
1-[2-(2,3-Dichloro-phenylsulfanyl)-ethyl]-4-(2,3-dihydro-benzo[1,4]dioxin-5-yl)-piperazine;
1-[2-(2-Allyl-6-chloro-phenoxy)-ethyl]-4-(2,3-dihydro-benzo[1,4]dioxin-5-yl)-piperazine;
1-(2,3-Dihydro-benzo[1,4]dioxin-5-yl)-4-[3-(2,4-dimethyl-phenylsulfanyl)-propyl]-piperazine;
1-(2,3-Dihydro-benzo[1,4]dioxin-5-yl)-4-[3-(2-trifluoromethyl-phenylsulfanyl)-propyl]-piperazine;
1-[3-(2,3-Dichloro-phenylsulfanyl)-propyl]-4-(2,3-dihydro-benzo[1,4]dioxin-5-yl)-piperazine;
1-[3-(3,4-Dichloro-phenylsulfanyl)-propyl]-4-(2,3-dihydro-benzo[1,4]dioxin-5-yl)-piperazine;
1-[4-(3,4-Dichloro-phenylsulfanyl)-butyl]-4-(2,3-dihydro-benzo[1,4]dioxin-5-yl)-piperazine;
1-[4-(2-Chloro-5-methyl-phenoxy)-butyl]-4-(2,3-dihydro-benzo[1,4]dioxin-5-yl)-piperazine;
1-[2-(2,4-Dichloro-phenylsulfanyl)-ethyl]-4-(2,3-dihydro-benzo[1,4]dioxin-5-yl)-piperazine;
1-(2,3-Dihydro-benzo[1,4]dioxin-5-yl)-4-(3-m-tolylsulfanyl-propyl)-piperazine;
1-[4-(2,4-Dichloro-phenylsulfanyl)-butyl]-4-(2,3-dihydro-benzo[1,4]dioxin-5-yl)-piperazine;
1-(2,3-Dihydro-benzo[1,4]dioxin-5-yl)-4-[2-(2-ethyl-phenylsulfanyl)-ethyl]-piperazine;
1-[2-(2,5-Dichloro-phenylsulfanyl)-ethyl]-4-(2,3-dihydro-benzo[1,4]dioxin-5-yl)-piperazine;
1-[2-(3-Chloro-phenylsulfanyl)-ethyl]-4-(2,3-dihydro-benzo[1,4]dioxin-5-yl)-piperazine;
1-[2-(2-Chloro-phenylsulfanyl)-ethyl]-4-(2,3-dihydro-benzo[1,4]dioxin-5-yl)-piperazine;
1-(2,3-Dihydro-benzo[1,4]dioxin-5-yl)-4-[2-(2-fluoro-phenylsulfanyl)-ethyl]-piperazine;
1-(2,3-Dihydro-benzo[1,4]dioxin-5-yl)-4-[3-(2-ethyl-phenylsulfanyl)-propyl]-piperazine;
1-[3-(2,5-Dichloro-phenylsulfanyl)-propyl]-4-(2,3-dihydro-benzo[1,4]dioxin-5-yl)-piperazine;
1-[3-(3-Chloro-phenylsulfanyl)-propyl]-4-(2,3-dihydro-benzo[1,4]dioxin-5-yl)-piperazine;
1-(2,3-Dihydro-benzo[1,4]dioxin-5-yl)-4-[3-(2-fluoro-phenylsulfanyl)-propyl]-piperazine;
3-Chloro-4-{4-[4-(2,3-dihydro-benzo[1,4]dioxin-5-yl)-piperazin-1-yl]-butoxy}-benzonitrile;
1-(2,3-Dihydro-benzo[1,4]dioxin-5-yl)-4-(4-o-tolylsulfanyl-butyl)-piperazine;
1-[4-(2,5-Dichloro-phenylsulfanyl)-butyl]-4-(2,3-dihydro-benzo[1,4]dioxin-5-yl)-piperazine;
1-[4-(2-Chloro-phenylsulfanyl)-butyl]-4-(2,3-dihydro-benzo[1,4]dioxin-5-yl)-piperazine;
1-(2,3-Dihydro-benzo[1,4]dioxin-5-yl)-4-[4-(2-fluoro-phenylsulfanyl)-butyl]-piperazine;
1-(2,3-Dihydro-benzo[1,4]dioxin-5-yl)-4-[2-(3-,4-dimethoxy-phenylsulfanyl)-ethyl]-piperazine;
3-{4-[4-(2,3-Dihydro-benzo[1,4]dioxin-5-yl)-piperazin-1-yl]-butoxy}-benzonitrile;
1-[4-(2-Chloro-4-fluoro-phenylsulfanyl)-butyl]-4-(2,3-dihydro-benzo[1,4]dioxin-5-yl)-piperazine;
1-(2,3-Dihydro-benzo[1,4]dioxin-5-yl)-4-[3-(4-trifluoromethoxy-phenylsulfanyl)-propyl]-piperazine;
1-(2,3-Dihydro-benzo[1,4]dioxin-5-yl)-4-[3-(2,5-dimethoxy-phenylsulfanyl)-propyl]-piperazine;
1-(2,3-Dihydro-benzo[1,4]dioxin-5-yl)-4-[3-(3-bromo-phenylsulfanyl)-propyl]-piperazine;
1-(2,3-Dihydro-benzo[1,4]dioxin-5-yl)-4-[4-(2-methoxy-phenylsulfanyl)-butyl]-piperazine;
1-(2,3-Dihydro-benzo[1,4]dioxin-5-yl)-4-[4-(2-isopropyl-phenylsulfanyl)-butyl]-piperazine;
1-(2,3-Dihydro-benzo[1,4]dioxin-5-yl)-4-(2-o-tolylsulfanyl-ethyl)-piperazine;
1-[4-(2-Allyl-phenoxy)-butyl]-4-(2,3-dihydro-benzo[1,4]dioxin-5-yl)-piperazine;
or an acid addition salt thereof.

14. A pharmaceutical composition comprising a compound according to claim 1 or a pharmaceutically acceptable acid addition salt thereof and at least one pharmaceutically acceptable carrier or diluent.

15. A method for the treatment of a disorder or disease of living animal body which is responsive to the effect of 5-HT_{1A} and D₄ receptors, said method comprising administering to said living animal body a therapeutically effective amount of a compound according to claim 1 or a pharmaceutically acceptable acid addition salt thereof.

16. The method of claim 15, wherein said living animal is a human.

17. The method of treatment of claim 16, wherein the disorder or disease is a neurological disorder.

18. The method of claim 17, wherein said neurological disorder is psychosis.

19. The method of claim 16, wherein said disorder or disease is an affective disorder selected from the group consisting of general anxiety disorder, panic disorder, obsessive compulsive disorder, depression, social phobia and eating disorders.
